(19) United States
(12) Patent Application Publication
(10) Pub. No.: US 2024/0360406 A1
(43) Pub. Date: Oct. 31, 2024

(54) METHODS AND COMPOSITION FOR TREATING NEURODEGENERATIVE DISEASES

(71) Applicant: Case Western Reserve University, Cleveland, OH (US)

(72) Inventors: Paul J. Tesar, Wickliffe, OH (US); Benjamin L.L. Clayton, University Heights, OH (US)

(21) Appl. No.: 18/687,438

(22) PCT Filed: Aug. 31, 2022
(86) PCT No.: PCT/US2022/042239

§ 371 Date: Feb. 28, 2024

Related U.S. Application Data
(60) Provisional application No. 63/239,627, filed on Sep. 1, 2021.

(51) Int. Cl. C12N 5/00; C12N 5/079

(52) U.S. Cl. CPC C12N 5/0037; C12N 5/0622; C12N 2500/32; C12N 2500/99; C12N 2501/115; C12N 2501/13; C12N 2501/15; C12N 2501/155; C12N 2501/2306; C12N 2501/25

(57) ABSTRACT

The invention described herein provides compositions and methods to maintain astrocytes in resting, enrichment and/or maturation, or activation states so that potential therapeutic agents treating diseases or conditions associated with reactive astrocytes can be identified.

### REFERENCE TO RELATED APPLICATION

This application claims priority to and the benefit of the filing date of U.S. Provisional Patent Application No. 63/239,627, filed on Sep. 1, 2021, the entire contents of which, including any sequence listing and drawings, are incorporated herein by reference.

### GOVERNMENT SUPPORT

This invention was made with government support under Grant No. R35NS116842, awarded by the National Institute of Health and National Institute of Neurological Disorders and Stroke. The government has certain rights in the invention.

### BACKGROUND OF THE INVENTION

Astrocytes are stellate cells in the central nervous system that perform a myriad of tasks that are necessary for the proper function of the brain.

Astrocytes are also involved in the neuroinflammatory response. Neuroinflammation refers to a state of reactivity of astrocytes and microglia induced by various pathological conditions, and may be associated with the recruitment of peripheral macrophages and lymphocytes. Reactive astrocytes and microglia mediate the innate immune responses in the brain. Astrocytes become reactive in response to virtually all pathological situations in the brain, both following acute injuries (stroke, trauma, axotomy, ischemia, infection, inflammation, traumatic brain injury), and during progressive diseases such as tumors, epilepsy, and neurodegenerative diseases (ND).

Here, astrocyte reactivity or reactive astrocytes refer to astrocytes that respond to any pathological condition in the CNS. Astrocytes are considered reactive when they become hypertrophic and overexpress the intermediate filament GFAP (glial fibrillary acidic protein)-two of the most universal hallmarks of reactivity. But this definition does not exclude many additional transcriptional, morphological and functional changes that occur in a disease-specific manner. In general, astrocyte reactivity involves the activation of transcriptional program(s) triggered by specific signaling cascades that results in long-lasting changes in morphology and function, persisting over several hours, days or even decades.

Astrocyte reactivity is not unique to human. It has been observed in many mammalian and bird species. Even in lampreys, newts and frogs, astrocyte-like cells react to injury and form a glial bridge promoting axonal regeneration. In Drosophila, glial cells with some typical astrocyte functions display strong phagocytic activity and morphological changes following neuronal degeneration.

Astrocyte reactivity was originally characterized by morphological changes—hypertrophy (enlarged cell body and processes), remodeling of processes, etc. It is also characterized by transcriptional and functional changes such as the overexpression of GFAP. However, it is unclear how the normal supportive functions of astrocytes are altered by their reactive state. In neurodegenerative diseases, for example, neuronal dysfunction and astrocyte reactivity take place over several years or even decades, making the issue even more complex and highly debated, with several conflicting reports published recently.

Regardless, research has shown that astrocyte reactivity is a shared and central feature in numerous neurodegenerative diseases, including Multiple Sclerosis (MS), Alzheimer's Disease (AD), Huntington's diseases (HD), Amyotrophic Lateral Sclerosis (ALS), and Parkinson's Disease (PD).

Astrocyte reactivity can be detected in the brain of AD patients with imaging and proteomic techniques even before the onset of symptoms. Foci of reactive astrocytes are also detected at early stages in some mouse models, even before amyloid deposition. Reactive astrocytes are usually found around amyloid plaques. Patches of reactive astrocytes may also be found in the absence of plaques in patients. In addition, atrophied astrocytes may be located at a distance from plaques in some mouse models.

Astrocyte reactivity is also an early feature of HD-GFAP immunoreactivity is detected in the striatum of presymptomatic carriers, and it increases with disease progression. Strikingly, no clear evidence of astrocyte reactivity exists in most HD models. Instead, HD astrocytes show functional alterations in the absence of the main features of reactivity—hypertrophy and high GFAP expression.

Reactive astrocytes are observed in both ALS patients and ALS models. They appear in vulnerable regions, and the degree of reactivity correlates with the level of neurodegeneration.

In PD, though the involvement of microglial cells in PD has been more extensively studied than that of astrocytes, astrocyte reactivity is detected in the SNpc (substantia nigra pars compacta) of patients with PD, individuals intoxicated with MPTP (1-methyl-4-phenyl-1,2,3,6-tetrahydropyridine) and in animal models.

Given the involvement of reactive astrocytes in numerous neurodegenerative diseases, there is a strong interest and utility in mimicking the transition of resting astrocytes to reactive astrocytes, especially in tissue culture or in vitro.

With the development of in vitro systems to study astrocytes in the 1980's, it became possible to study reactive astrocytes in culture. Human astrocytes can be cultured in vitro, either from fetuses or biopsies, or be generated from induced pluripotent stem cells, including from patients. Generally, primary astrocytes are exposed to cytokines such as interleukins (IL), tumor necrosis factor alpha (TNFα) and interferon gamma (IFNγ), which induce many transcriptional and functional changes in astrocytes.

The main limitation to such in vitro studies is that astrocytes in a dish show signs of reactivity, even in the absence of stimulus. They express high levels of GFAP, and usually have a flat, polygonal morphology, very different from the bushy or stellate morphology observed in situ. This precludes the identification of the hallmarks of astrocyte reactivity. Although astrocytes can be co-cultured with neurons to maintain their stellate morphology and low GFAP expression, presumably due to uncharacterized factors released by neurons, such co-cultures are not pure population of resting astrocytes for further study, and the uncharacterized neuronal factors interfere with the stimulation of resting astrocytes to reactive astrocytes.

Further, existing methods generate astrocytes based on two main procedures—astrocytes are either enriched from mixed cell culture by taking advantage of their ability to strongly adhere to tissue culture plastic by shaking off other cell types, or astrocyte specific antibodies are used to prospectively isolate astrocytes from mixed cell suspension by fluorescence, magnetic, or immunopanning sorting techniques. Both approaches have benefits and drawbacks. The “shake-off” method has insufficient purity, and requires the addition of serum which contains unknown components and does not normally reach astrocytes in the brain. The prospective isolation via astrocyte-specific antibodies avoids the problems associated with the “shake-off” method, but cannot scale to the cell numbers needed for certain assays including high-throughput drug screening.

Other methods have recently been developed to reduce astrocyte reactivity in vitro, such as exposure to heparin-binding EGF-like growth factor, or 3D polymer matrix. However, due to the various limitations described above, most studies about reactive astrocytes are still obtained in expensive and inconvenient animal models.

Thus, there is a need for a system and a method to isolate and culture resting astrocytes in their resting state, until desired stimulation to reactive astrocyte is induced. Such methods and systems can be used to isolate, enrich, and expand astrocytes to overcome the above-discussed issues, and would greatly benefit the study of astrocytes and discovery of potential therapeutic agents for treating diseases associated with reactive astrocytes.

### SUMMARY OF THE INVENTION

One aspect of the invention provides a chemically-defined, serum-free, resting astrocyte culture medium, for culturing resting astrocytes, the medium comprising: i) a serum-free basal medium (such as DMEM/Neurobasal medium), wherein said basal medium (1) is devoid of significant source of proteins, lipids, or growth factors, and/or (2) comprises sufficient energy source, nitrogen source, carbon source, amino acids, vitamins, an inorganic salts to support growth of mammalian neuronal cells in the absence of feeder cells, optionally, the serum-free basal medium further comprises (3) an amino acid supplement as a source of L-glutamine (such as GLUTAMAX™ brand of L-alanyl-L-glutamine dipeptide), such as about 1-3 mM (e.g., about 2 mM) L-alanyl-L-glutamine dipeptide; and/or a source of sodium pyruvate, such as about 0.5-1.5 mM (e.g., about 1 mM) sodium pyruvate, ii) an antioxidant (e.g., including one that inhibits lipid peroxidation, such as sodium selenite); iii) an iron carrier that regulates iron homeostasis (e.g., transferrin); iv) a polyamine that promotes cell division (e.g., putrescine); v) a hormone that activates the progesterone receptor (e.g., progesterone); and, vi) a trophic factor that promotes astrocyte survival in culture; wherein resting astrocytes cultured in said resting astrocyte culture medium exhibit a stellate morphology, express canonical mature astrocyte markers (e.g., AQP4, GLT-1, VIMENTIN, GLAST, and/or ALDH1L1), and/or do not express reactive astrocyte markers (e.g., Lcn2, Steap4, and Cxc110).

In certain embodiments, the trophic factor that promotes astrocyte survival is a neurotrophic factor.

In certain embodiments, the neurotrophic factor is selected from the group consisting of heparin binding EGF like growth factor (HBEGF), nerve growth factor (NGF), brain-derived neurotrophic factor (BDNF), neurotrophin-3 (NT-3), and ciliary neurotrophic factor (CNTF).

In certain embodiments, the neurotrophic factor is HBEGF; optionally, about 2-10 ng/mL (e.g., about 5 ng/mL) HBEGF.

In certain embodiments, the antioxidant is selected from the group consisting of sodium selenite and N-acetylcysteine (NAC), catalase, reduced glutathione, alpha-tocopherol, and superoxide dismutase.

In certain embodiments, the antioxidant comprises NAC and sodium selenite; optionally, about 20-40 μM (e.g., about 30 μM) NAC and/or about 5.2-40 ng/mL (e.g., about 10-20 ng/mL) sodium selenite.

In certain embodiments, the serum-free basal medium further comprises (3a) an amino acid supplement as a source of L-glutamine (such as GLUTAMAX™ brand of L-alanyl-L-glutamine dipeptide), such as about 1-3 mM (e.g., about 2 mM) L-alanyl-L-glutamine dipeptide; and/or, (3b) a source of sodium pyruvate, such as about 0.5-1.5 mM (e.g., about 1 mM) sodium pyruvate.

In certain embodiments, the serum-free basal medium further comprising (4) a growth factor that facilitates utilization of glucose and amino acids (e.g., insulin or insulin like growth factor 1 (IGF-1)); optionally, about 0-25 pg/mL insulin.

In certain embodiments, the resting astrocyte culture medium of various embodiments of the above aspects or any other aspect of the invention delineated herein comprises i) 1:1 mixture of DMEM/Neurobasal media mixture comprising sodium pyruvate and L-alanyl-L-glutamine dipeptide in the serum-free basal medium; ii) sodium selenite and NAC as said antioxidant; iii) transferrin as said iron carrier; iv) putrescine as said polyamine; v) progesterone as said hormone that activates the progesterone receptor; and, vi) HBEGF as said trophic factor that promotes astrocyte survival in culture.

In certain embodiments, the resting astrocyte culture medium of various embodiments of the above aspects or any other aspect of the invention delineated herein comprises i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture; ii) about 5.2-40 ng/mL (e.g., about 10-20 ng/mL) sodium selenite and about 20-40 μM (e.g., about 30 μM) NAC; iii) about 50-200 pg/mL (e.g., about 100 pg/mL) transferrin; iv) about 8-32 pg/mL (e.g., about 16 pg/mL) putrescine; v) about 6-60 ng/mL (e.g., about 30 ng/mL) progesterone; vi) about 0-25 pg/mL insulin; and, vii) about 5-10 ng/mL (e.g., about 5 ng/mL) HBEGF.

Another aspect of the invention provides a chemically-defined, serum-free, astrocyte enrichment and/or maturation culture medium, for astrocyte enrichment and/or maturation, the medium comprising the resting astrocyte culture medium of various embodiments of the above aspects or any other aspect of the invention delineated herein, and further comprising: viii) a TGF-beta superfamily cytokine that promotes astrogenesis and astrocyte maturation through SMAD dependent-signaling; ix) an IL-6 superfamily cytokine that actives STAT signaling through leukemia inhibitory factor (LIF) receptor R (LIFRO) and/or glycoprotein 130 (gp130); and x) a mitogen and trophic factor that promotes astrocyte survival and proliferation, as well as a resting/quiescent astrocyte state; wherein said cell culture medium promotes enrichment and/or maturation for astrocytes in astrocyte-containing neuronal tissues.

In certain embodiments, the factor to activate SMAD dependent-signaling is a transforming growth factor β (TGF-β) family member.

In certain embodiments, wherein the TGF-P family member is a bone morphogenetic protein (BMP).

In certain embodiments, the BMP is selected from the group consisting of BMP2, BMP4, BMP5, BMP6, BMP7, BMP10 and BMP15.

In certain embodiments, the BMP is BMP4; optionally, 5-100 ng/mL BMP4 (e.g., 5-ng/mL BMP4 for astrocyte enrichment in said astrocyte enrichment and/or maturation culture medium; and/or about 25-100 ng/mL BMP4 for astrocyte maturation in said astrocyte enrichment and/or maturation culture medium).

In certain embodiments, the IL-6 superfamily cytokine is IL-6, ciliary neurotrophic factor (CNTF), leukemia inhibitory factor (LIF), or oncostatin M (OSM).

In certain embodiments, the IL-6 superfamily cytokine is CNTF; optionally, about 5-20 ng/mL (e.g., about 10 ng/mL) CNTF.

In certain embodiments, the mitogen and trophic factor is epidermal growth factor (EGF), basic fibroblast growth factor (bFGF), fibroblast growth factor 2 (FGF2), fibroblast growth factor 8 (FGF8), platelet derived growth factor (PDGF), and insulin like growth factor 1 (IGF-1).

In certain embodiments, the mitogen and trophic factor is FGF2; optionally about 10-40 ng/mL (about 20 ng/mL) FGF2.

In certain embodiments, the astrocyte enrichment and/or maturation culture medium of various embodiments of the above aspects or any other aspect of the invention delineated herein comprises: i) 1:1 mixture of DMEM/Neurobasal media mixture comprising sodium pyruvate and L-alanyl-L-glutamine dipeptide in the serum-free basal medium; ii) sodium selenite and NAC as said antioxidant; iii) transferrin as said iron carrier; iv) putrescine as said polyamine; v) progesterone as said hormone that activates the progesterone receptor; vi) HBEGF as said trophic factor that promotes astrocyte survival in culture; vii) BMP4 as the TGF-beta superfamily cytokine; viii) CNTF as the IL-6 superfamily cytokine; and, ix) FGF2 as the mitogen and trophic factor.

In certain embodiments, the astrocyte enrichment and/or maturation culture medium of various embodiments of the above aspects or any other aspect of the invention delineated herein comprises: i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture; ii) about 5.2-40 ng/mL (e.g., about 10-20 ng/mL) sodium selenite and about 20-40 μM (e.g., about 30 μM) NAC; iii) about 50-200 pg/mL (e.g., about 100 pg/mL) transferrin; iv) about 8-32 pg/mL (e.g., about 16 pg/mL) putrescine; v) about 6-60 ng/mL (e.g., about 30 ng/mL) progesterone; vi) about 0-25 pg/mL insulin; vii) about 2-10 ng/mL (e.g., about 5 ng/mL) HBEGF; viii) about 5-20 ng/mL (e.g., about 10 ng/mL) CNTF; ix) about 10-40 ng/mL (about 20 ng/mL) FGF2; and, x) about 5-20 ng/mL (e.g., about 10 ng/mL) BMP4.

In certain embodiments, the astrocyte enrichment and/or maturation culture medium of various embodiments of the above aspects or any other aspect of the invention delineated herein comprises: i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture; ii) about 5.2-40 ng/mL (e.g., about 10-20 ng/mL) sodium selenite and about 100-300 μM (e.g., about 200 μM) NAC; iii) about 50-200 pg/mL (e.g., about 100 pg/mL) transferrin; iv) about 8-32 pg/mL (e.g., about 16 pg/mL) putrescine; v) about 6-60 ng/mL (e.g., about 30 ng/mL) progesterone; vi) about 0-25 pg/mL insulin; vii) about 2-10 ng/mL (e.g., about 5 ng/mL) HBEGF; viii) about 5-20 ng/mL (e.g., about 10 ng/mL) CNTF; ix) about 10-40 ng/mL (about 20 ng/mL) FGF2; and, x) about 25-100 ng/mL (e.g., about 50 ng/mL) BMP4.

Another aspect of the invention provides a chemically-defined, serum-free, astrocyte activation culture medium, for transitioning astrocytes from resting state to activation state, the medium comprising the resting astrocyte culture medium of various embodiments of the above aspects or any other aspect of the invention delineated herein, except for the trophic factor that promotes astrocyte survival, and further comprising one or more microglia-derived reactive astrocyte drivers (or proinflammatory cytokines) that promote the transition of astrocytes to a damaging reactive state.

In certain embodiments, the reactive astrocyte drivers (or proinflammatory cytokines) comprise tumor necrosis factor alpha (TNFα), interleukin 1 alpha (IL1a), and/or complement component 1q (C1q).

In certain embodiments, the astrocyte activation culture medium of the above aspects or any other aspect of the invention delineated herein comprises: i) 1:1 mixture of DMEM/Neurobasal media mixture comprising sodium pyruvate and L-alanyl-L-glutamine dipeptide in the serum-free basal medium; ii) sodium selenite and NAC as said antioxidant; iii) transferrin as said iron carrier; iv) putrescine as said mitogen; v) progesterone as said hormone that activates the progesterone receptor; and, vi) TNFα, IL1a, and/or C1q as said reactive astrocyte drivers.

In certain embodiments, the astrocyte activation culture medium of the above aspects or any other aspect of the invention delineated herein comprises: i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture; ii) about 5.2-40 ng/mL (e.g., about 10-20 ng/mL) sodium selenite and about 20-40 μM (e.g., about 30 μM) NAC; iii) about 50-200 μg/mL (e.g., about 100 μg/mL) transferrin; iv) about 8-32 μg/mL (e.g., about 16 μg/mL) putrescine; v) about 6-60 ng/mL (e.g., about 30 ng/mL) progesterone; vi) about 0-25 μg/mL insulin; and, vii) about 1-5 ng/mL (e.g., about 3 ng/mL) IL-1α, about 15-60 ng/mL (e.g., about 30 ng/mL) TNFα, and about 200-800 ng/mL (e.g., about 400 ng/mL) C1q.

Another aspect of the invention provides a method of isolating a substantially pure culture of resting astrocytes from a population of astrocyte-containing cells, the method comprising: (1) culturing single cells of the population of astrocyte-containing cells, in said astrocyte enrichment culture medium of various embodiments of the above aspects or any other aspect of the invention delineated herein, with fresh medium change about every 2 days until a confluent culture is formed, in order to enrich for proliferating/proliferated astrocytes in the single cell culture; (2) passaging the confluent culture once in said astrocyte maturation culture medium of various embodiments of the above aspects or any other aspect of the invention delineated herein for about 2 days to permit astrocyte maturation, or optionally cryopreserving the confluent culture before said passaging; and, (3) replacing the astrocyte maturation culture medium after about 2 days, with said resting astrocyte culture medium of various embodiments of the above aspects or any other aspect of the invention delineated herein for about 3 more days, thereby producing the substantially pure culture of resting astrocytes.

In certain embodiments, the population of astrocyte-containing cells are derived from mammalian neuronal tissues, and differentiated or differentiating stem cells or progenitor cells.

In certain embodiments, the mammalian neuronal tissues are isolated from a central nervous system (CNS) tissue, such as cortex, corpus callosum, hippocampus, midbrain, pons, medulla, brainstem, cerebellum, and/or spinal cord.

In certain embodiments, said differentiated or differentiating stem cells or progenitor cells comprise oligodendrocyte progenitors (OPCs), glial progenitors (GPCs), glial restricted progenitors (GRPs), and/or neural progenitors (NPCs). In certain embodiments, the stem cells or progenitor cells comprise induced pluripotent stem cells (iPSCs), embryonic stem cells (ESCs), neural stem cells (NSCs), and/or epiblast stem cells (EpiSCs).

In certain embodiments, the astrocyte-containing cells are derived from mammalian neuronal tissues by chemical, enzymatic, and/or mechanical dissociation of the mammalian neuronal tissues.

In certain embodiments, said dissociation comprises dissociating the mammalian neuronal tissues using enzymatic digestion with mechanical trituration.

In certain embodiments, said enzymatic digestion is performed with papain, trypsin, dispase, and/or collagenase.

In certain embodiments, said resting astrocytes: (1) are positive for any one or more of the astrocyte markers selected from GFAP, AQP4, GLT-1, VIMENTIN, GLAST, and ALDH1L1; (2) do not express Lcn2, Steap4, and/or Cxc110; (3) exhibit a stellate morphology; and/or (4) only express mature astrocyte genes (such as Gja1 or Sox9), with no expression of oligodendrocyte genes (such as Sox10 and Mbp), microglia genes (such as Cd68 and Tmem119), and neuron markers (such as Nef1 and Snap25).

In certain embodiments, said resting astrocytes take up exogenous glutamate. In certain embodiments, said resting astrocyte take up at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of exogenous glutamate.

In certain embodiments, said resting astrocytes have increased phagocytic activity when compared to reactive astrocytes. In certain embodiments, said resting astrocytes have at least 1.2 fold, at least 1.3 fold, at least 1.4 fold, at least 1.5 fold, at least 1.6 fold, at least 1.7 fold, at least 1.8 fold, at least 1.9 fold, at least 2.0 fold increased phagocytic activity when compared to reactive astrocytes.

In certain embodiments, said resting astrocytes express one or more reactive astrocyte markers when infected with Thieler's murine encephalomyelitis virus (TMEV), thereby becoming reactive astrocytes. In certain embodiments, the reactive astrocyte marker is guanylate binding protein 2 (GBP2). In certain embodiments, said resting astrocytes infected with TMEV express at least 70%, at least 75%, at least 80%, at least 85%, at least 90%, at least 95%, or at least 98% more reactive astrocyte marker compared to uninfected resting astrocytes.

In certain embodiments, the method has at least about 90%, 95%, 96%, 97%, 98%, 99% or more of the cells in the substantially pure culture are resting astrocytes.

In certain embodiments, the substantially pure culture of resting astrocytes comprises about 10⁶, 10⁷, 10⁸, 10⁹, 10¹⁰, 10¹¹, 10¹² or 10¹³ resting astrocytes.

In certain embodiments, the method further comprises, before culturing the single cell culture in said astrocyte enrichment culture medium (such as the astrocyte enrichment culture medium of various embodiments of the above aspects or any other aspect of the invention delineated herein), culturing the single cells for about 24 hours in a media consisting of Dulbecco's Modified Eagle Medium/Nutrient Mixture F-12 (DMEM/F-12), N-2 Supplement, B-27 Supplement, GLUTAMAX™ Supplement, Penicillin-Streptomycin, and FGF-2.

Another aspect of the invention provides a cell culture comprising a population of astrocyte-containing cells cultured in the culture medium of various embodiments of the above aspects or any other aspect of the invention delineated herein.

In certain embodiments, the culture medium is (1) the chemically-defined, serum-free, astrocyte enrichment culture medium of various embodiments of the above aspects or any other aspect of the invention delineated herein; (2) the chemically-defined, serum-free, astrocyte maturation culture medium of various embodiments of the above aspects or any other aspect of the invention delineated herein; or, (3) the chemically-defined, serum-free, the resting astrocyte culture medium of various embodiments of the above aspects or any other aspect of the invention delineated herein.

Another aspect of the invention provides a cryopreserved culture of enriched astrocytes, obtained from a population of astrocyte-containing cells by: (a) culturing single cells of the population of astrocyte-containing cells, in said astrocyte enrichment culture medium of various embodiments of the above aspects or any other aspect of the invention delineated herein, with fresh medium change about every 2 days until a confluent culture is formed, in order to enrich for proliferating/proliferated astrocytes in the single cell culture; and, (b) cryopreserving the confluent culture.

Another aspect of the invention provides a cryopreserved substantially pure culture of resting astrocytes, wherein the substantially pure culture of resting astrocytes are obtained by the method of various embodiments of the above aspects or any other aspect of the invention delineated herein.

In certain embodiments, the population of astrocyte-containing cells are derived from mammalian neuronal tissues, and differentiated or differentiating stem cells or progenitor cells.

In certain embodiments, the mammalian neuronal tissues are isolated from a central nervous system (CNS) tissue, such as cortex, corpus callosum, hippocampus, midbrain, pons, medulla, brainstem, cerebellum, and/or spinal cord.

In certain embodiments, said differentiated or differentiating stem cells or progenitor cells comprise oligodendrocyte progenitors (OPCs), glial progenitors (GPCs), glial restricted progenitors (GRPs), and/or neural progenitors (NPCs).

In certain embodiments, the stem cells or progenitor cells comprise induced pluripotent stem cells (iPSCs), embryonic stem cells (ESCs), neural stem cells (NSCs), and/or epiblast stem cells (EpiSCs).

Another aspect of the invention provides a method of identifying a compound that inhibits reactive astrocyte formation from resting astrocytes, the method comprising: contacting a substantially pure culture of resting astrocytes obtained by the method of various embodiments of the above aspects or any other aspect of the invention delineated herein, or a thawed culture of the cryopreserved substantially pure culture of resting astrocytes of various embodiments of the above aspects or any other aspect of the invention delineated herein, with a candidate compound from a library of compounds, in the presence of the astrocyte activation culture medium of various embodiments of the above aspects or any other aspect of the invention delineated herein, for a sufficient period of time (e.g., 24 hours), before determining the expression level of a marker gene for reactive astrocyte; wherein the candidate compound that statistically significantly decreased the number of marker gene-positive reactive astrocytes by greater than 50%, 60%, 70%, 80%, 90%, 95% or more compared to vehicle (e.g., a solvent for the candidate compound such as DMSO) control is identified as the compound that inhibits reactive astrocyte formation.

In certain embodiments, the marker gene comprises one or more of GBP2, PSMB8, C3, H2-D1, H2-T23, SERPING1, and IIGP1.

In certain embodiments, the method further comprising determining the expression level of the marker gene.

In certain embodiments, the candidate compound is not significantly toxic to astrocytes (e.g., the candidate compound does not decrease the counted number of live cells by greater than 30% compared to the vehicle control).

In certain embodiments, the method has a z-prime score of 0.6, 0.7 or higher.

In certain embodiments, the resting astrocytes are contacted in 384-well tissue culture plates in a high throughput platform suitable for multiplex screening.

In certain embodiments, the resting astrocytes are contacted by the candidate compound before prior to (e.g., 1 hour prior to), simultaneously with, or subsequent to (e.g., within 30 min of) contacting with the astrocyte activation culture medium.

In certain embodiments, the astrocyte activation culture medium comprises about 1-5 ng/mL (e.g., about 3 ng/mL) IL-1α, about 15-60 ng/mL (e.g., about 30 ng/mL) TNFα, and about 200-800 ng/mL (e.g., about 400 ng/mL) C1q.

In certain embodiments, the method further comprising confirming that the candidate compound does not substantially affect (e.g., decrease) the expression level of a pan-astrocyte marker (such as vimentin).

In certain embodiments, the library of compounds comprise histone deacetylase (HDAC) inhibitors, proteasome inhibitors, and inhibitors of NFκB signaling.

It should be understood that any one embodiment of the invention described herein, including those described only in the examples or in the claims, can be combined with any one or more additional embodiments of the invention unless expressly disclaimed or being improper.

### BRIEF DESCRIPTION OF THE DRAWINGS

FIG. 1 depicts graphical representation of resting astrocyte isolation and enrichment. Cortices from mouse at postnatal day 2-4 (P2-4) are removed and chemically dissociated at 37° C. using commercially available dissociation kits (Miltenyi, Cat #: 130-095-929) followed by mechanical dissociation to generate a single cell suspension that is plated and then enriched for astrocytes using astrogenic factors HBEGF, CNTF, BMP4, and FGF2.

FIG. 2 depicts phase contrast and immunofluorescence of resting astrocytes. Resting astrocytes exhibit a stellate morphology similar to their in vivo morphology. Resting astrocytes express canonical mature astrocyte markers GFAP, AQP4, GLT-1, and ALDH1L1.

FIG. 3 depicts resting astrocyte cultures are highly pure as shown by single-cell RNAseq. Single-cell RNAseq of resting astrocyte cultures show that only mature astrocyte genes are expressed with no expression of oligodendrocyte, microglia, or neuron markers.

FIG. 4 depicts graphical representation of primary screen for inhibitors of damaging reactive astrocyte polarization. Astrocytes are plated onto 384 well plates and cultured for 2-days in astrocyte maturation media, followed by 3-days in resting astrocyte media. Chemical compound was then added 1 hr prior to adding proinflammatory cytokines TNFα, IL1α, and C1q (TIC). After 24 hrs of incubation cells were fixed and stained with GBP2, a marker for damaging reactive astrocytes, and vimentin, a pan astrocyte marker, and then imaged using a Perkin Elmer Operetta High-Content imaging system.

FIG. 5 shows an example of an ineffective negative compound and an effective hit compound. A non-toxic ineffective negative compound does not decrease the expression of guanylate binding protein 2 (GBP2) in astrocytes exposed to reactive astrocyte media. An effective hit compound decreases the expression of GBP2 in astrocytes exposed to reactive astrocyte media, is not-toxic and does not decrease the total number of cells in the well by greater than 30% compared to control wells, and does not affect the expression of the counterstain for vimentin (VIM).

FIG. 6 depicts a dot plot representation of primary screen results. Each gray dot represents a non-toxic compound that was not validated as an inhibitor of reactive astrocyte formation. Hit compounds in black were defined as those that decreased the formation of GBP2+ reactive astrocytes by 80% or more and were further validated in secondary dose curve assays.

FIG. 7 is a bar graph showing glutamate uptake in resting astrocytes. Resting astrocytes cultured in glutamate free media were exposed to a known concentration of exogenous glutamate, incubated for 6 hrs, before the amount of glutamate uptake by the resting astrocytes was measured.

FIG. 8 shows phagocytosis activity of resting astrocytes and reactive astrocytes. Resting and reactive astrocytes were exposed to fluorescently-labelled myelin debris. After 24 hrs, myelin debris phagocytosed by astrocytes was measured.

FIG. 9 shows that resting astrocytes infected with TMEV became reactive. Resting astrocytes were exposed to TMEV at two different multiplicity of infection (MOI) for 1 hr. After 1 hr, virus was washed out before 24 hrs of incubation and staining for double-stranded The nucleic acid signal represents a measure for viral infection, and the reactive astrocytes are marked by the GBP2 marker.

FIG. 10 shows reactive astrocytes inhibit the formation of mature oligodendrocytes in co-culture. Resting or reactive astrocytes were co-cultured with differentiating oligodendrocytes for 48 to 72 hrs and then stained for the mature oligodendrocyte markers 01 or MBP.

### DETAILED DESCRIPTION OF THE INVENTION

### 1. Overview

The invention described herein provides compositions, e.g., various serum-free, chemically defined tissue culture media, useful for the isolation, enrichment, maturation, and/or activation of astrocytes, including primary astrocytes (astrocytes isolated from primary tissues), astrocytes differentiated from pluripotent stem cells or oligodendrocyte progenitor cells (OPCs), or reprogramed somatic cells. The astrocyte enrichment and/or maturation culture medium of the invention can be used to enrich and purify astrocytes from such primary astrocytes, differentiated astrocytes, or reprogramed astrocytes. The resting astrocyte culture medium of the invention can be used to culture and maintain the isolated/purified resting astrocytes under conventional tissue culture conditions. The astrocyte activation culture medium of the invention can be used to stimulate or transition cultured resting astrocytes to reactive astrocytes, to study the reactive astrocytes, their transition from resting astrocytes, and compounds that inhibit this process as potential therapeutic agents for numerous diseases associated with reactive astrocytes, including the various neurodegenerative diseases (NDs).

The invention is partly based on the discovery that, using the formulation of the subject astrocyte enrichment and/or maturation culture medium, a highly purified astrocyte population can be isolated or purified from a mixture of cell types, including astrocytes and other non-astrocyte cells normally found with astrocytes (such as neurons from neuronal tissues). Thus, in some embodiments, the culture medium of the invention is an enrichment and/or maturation astrocyte culture media capable of promoting enrichment and/or maturation for astrocytes in astrocyte-containing primary neuronal tissues.

The invention is also partly based on the discovery that such isolated/purified astrocytes can be maintained in the resting astrocyte culture medium of the invention, and exist in conventional tissue culture conditions, without losing their hallmark stellate morphology, and without expressing reactive astrocyte markers such as Lcn2, Steap4, and Cxc110. The resting astrocytes can be cryopreserved for long term storage, and can be thawed and maintained as resting astrocytes in the resting astrocyte culture medium of the invention. Thus, in some embodiments, the culture medium of the invention is a resting astrocyte culture media capable of maintaining astrocytes in resting state.

The invention is further partly based on the discovery that, astrocytes cultured in media to maintain their resting state can stimulated by one or more microglia-derived reactive astrocyte drivers or proinflammatory cytokines (e.g., TNFα, IL1α, and/or C1q), that promote the transition of astrocytes to a damaging reactive state (reactive astrocyte). This process can be used to screen for therapeutic agents that are capable of inhibiting the formation of damaging reactive state astrocytes. Thus, in some embodiments, the culture medium of the invention is an astrocyte activation media capable of promoting the transition of astrocytes to a damaging reactive state.

More specifically, the invention described herein provides media and methods for the generation of highly pure in vitro cultures of resting astrocytes that can be grown as resting astrocytes indefinitely, in chemically defined serum-free media, without losing their stellate morphology and without expressing common reactive astrocyte markers such as Lcn2, Steap4, and Cxc110.

This technology can be used to obtain astrocytes from numerous astrocyte-containing tissues or cell mixtures, such as mammalian primary brain tissue, astrocytes differentiated from progenitor cells including oligodendrocyte progenitors (OPCs), glial progenitors (GPCs), glial restricted progenitors (GRPs), and induced pluripotent stem cells (iPSCs), embryonic stem cells (ESCs), and epiblast stem cells (EpiSCs). This technology improves upon existing technologies by generating scalable resting astrocytes suitable for high-throughput drug screening, without the use of serum. This technology also generates astrocytes in an unbiased fashion without the required use of astrocyte specific antibody capture which may preferentially isolate a specific astrocyte subtype.

One specific use of the generated resting astrocytes is for drug screening. As mentioned above, astrocytes respond to brain injury and play vital roles in the pathogenesis of neurodegenerative diseases. In response to brain injury including, inflammation, traumatic brain injury, stroke, and neurodegenerative diseases including multiple sclerosis, Alzheimer's disease, Huntington's disease, Parkinson's disease, and amyotrophic lateral sclerosis, astrocytes can become polarized and play either a beneficial or damaging role. Astrocytes that polarize to a damaging reactive state contribute to brain damage and represent a potential therapeutic target. Agents inhibiting the transition from resting astrocytes to reactive astrocytes are potential therapeutic agents that can be used to treat reactive-astrocytes associated diseases, including neurodegenerative diseases such as MS, AD, HD, PD, and ALS.

With the general aspects of the invention described herein, more detailed aspects of the invention are provided in further sections below.

### II. Definitions

As used herein, “around,” “about,” or “approximately” shall generally mean within 20 percent, preferably within 10 percent, preferably within 5 percent, and more preferably within 1 percent of a given value or range. Numerical quantities given herein are approximate, meaning that the term “around,” “about,” or “approximately” can be inferred if not expressly stated.

The term “serum-free medium” refers to a serum-free medium that is devoid of animal serum—a chemically undefined composition that contains many important factors for tissue culture in vitro.

A “serum free basal medium” is a serum-free medium with defined chemical composition, and is devoid of significant source of proteins, lipids, or growth factors, but comprises sufficient energy source, nitrogen source, carbon source, amino acids, vitamins, and inorganic salts to support growth of mammalian neuronal cells in the absence of feeder cells. In certain embodiments, the serum-free basal medium further comprises an amino acid supplement as a source of L-glutamine, such as L-glutamine or LUTAMAX™ brand of L-alanyl-L-glutamine dipeptide, which can release L-glutamine in tissue culture. In certain embodiments, the serum-free basal medium further comprises a source of sodium pyruvate.

Examples of serum-free basal media include, but are not limited to, DMEM and Neurobasal media, or mixture thereof. One exemplary serum-free basal medium of the invention is 1:1 mixture of DMEM/Neurobasal media mixture comprising sodium pyruvate and L-alanyl-L-glutamine dipeptide.

As used herein, the term “amino acid supplement” refers to any amino acid supplement that can be added to media to provide amino acids as an energy source, source of nitrogen, or for synthesis of proteins and/or nucleic acids. For example, amino acid supplement can be L-glutamine, which serves as an auxiliary energy source. An alternative L-glutamine source is commercial product such as GLUTAMAX™ brand of L-alanyl-L-glutamine dipeptide, which can release L-glutamine in tissue culture.

As used herein, the term “carbon source” includes a supplement added to a cell culture media to supply a source of carbon for the cell (e.g., for making organic compounds with carbon atoms). A non-limiting example of carbon source is sodium pyruvate.

As used herein, the term “antioxidant” is any substance that inhibits oxidation, usually because it is preferentially oxidized itself. In some embodiments, the antioxidant inhibits lipid peroxidation. Examples of antioxidants include, but are not limited to, sodium selenite, N-acetylcysteine (NAC), catalase, reduced glutathione, alpha-tocopherol, superoxide dismutase, and mixture thereof, such as NAC and sodium selenite.

As used herein, the term “iron carrier” includes a molecule that regulates iron homeostasis in cells. A non-limiting example of an iron carrier is transferrin.

As used herein, the term “hormone that activates the progesterone receptor” refers to a molecule that activated the progesterone receptor that promotes cell growth. A non-limiting example of said hormone is progesterone.

As used herein, the term “trophic factor” refers to a molecule that promotes survival of cells (e.g., astrocytes) in culture. In some embodiments, the trophic factor is a neurotrophic factor. For example, a trophic factor includes, but not limited to, heparin binding EGF like growth factor (HBEGF), nerve growth factor (NGF), brain-derived neurotrophic factor (BDNF), neurotrophin-3 (NT-3), and ciliary neurotrophic factor (CNTF). In some embodiments, the trophic factor is HBEGF.

As used herein, the term “growth factor that facilitates utilization of glucose and amino acids” includes a molecule that facilitates utilization of glucose and amino acids. For example, a growth factor that facilitates utilization of glucose and amino acids can be insulin or insulin like growth factor 1 (IGF-1).

As used herein, the term “polyamine” refers to an organic compound having more than two amino groups. In some embodiments, the polyamine promotes proper cell division. For example, but not limited to, a polyamine can be putrescine.

As used herein, the term “IL-6 subfamily member” or “IL-6 superfamily cytokine” refers to a protein capable of activating STAT signaling through leukemia inhibitory factor (LIF) receptor R (LIFRO) and/or glycoprotein 130 (gp130). For example, an IL-6 superfamily cytokine includes, but is not limited to, IL-6, ciliary neurotrophic factor (CNTF), leukemia inhibitory factor (LIF), or oncostatin M (OSM).

As used herein, the term “TGF-beta superfamily cytokine” or “TGF-beta superfamily member” refers to a protein capable of activating SMAD dependent-signaling thereby promoting astrogenesis and astrocyte maturation. For example, a TGF-beta superfamily cytokine or member includes, but is not limited to, transforming growth factor R (TGF-P) family member, e.g., bone morphogenetic protein (BMP), e.g., BMP2, BMP4, BMP5, BMP6, BMP7, BMP10 and BMP15.

As used herein, the term “mitogen and trophic factor” refers to a protein that promotes astrocyte survival and proliferation, as well as a resting/quiescent astrocyte state. For example, a mitogen and trophic factor can include, but is not limited to, epidermal growth factor (EGF), basic fibroblast growth factor (bFGF), fibroblast growth factor 2 (FGF2), fibroblast growth factor 8 (FGF8), platelet derived growth factor (PDGF), and insulin like growth factor 1 (IGF-1). In some embodiments, the mitogen and trophic factor is FGF2.

As used herein, the term “differentiated cell” includes any primary cell that is not, in its native form, pluripotent as that term is defined herein. The term “differentiated cell” refers to a cell of a more specialized cell type derived from a cell of a less specialized cell type (e.g., a stem cell such as an induced pluripotent stem cell) in a cellular differentiation process.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell type: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type.

Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e g, induced or by complete reversal) from a non-pluripotent cell, typically an adult somatic cell, for example, by inducing a forced expression of one or more genes.

The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stemness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

### III. Culture Media

In some embodiments, the culture media is a resting astrocyte culture media capable of maintaining astrocytes in resting state. In some embodiments, the resting astrocyte culture media comprises a serum-free basal medium, an antioxidant, an iron carrier that regulates iron homeostasis, a polyamine that promotes cell division, a hormone that activates the progesterone receptor, and a trophic factor that promotes astrocyte survival in culture.

In some embodiments, the culture media is an enrichment and/or maturation astrocyte culture media capable of promoting enrichment and/or maturation for astrocytes in astrocyte-containing primary neuronal tissues. In some embodiments, the enrichment and/or maturation culture medium comprises a serum-free basal medium, an antioxidant, an iron carrier that regulates iron homeostasis, a polyamine that promotes cell division, a hormone that activates the progesterone receptor, a trophic factor that promotes astrocyte survival in culture, a TGF-beta superfamily cytokine that promotes astrogenesis and astrocyte maturation through SMAD dependent-signaling, an IL-6 superfamily cytokine that actives STAT signaling through leukemia inhibitory factor (LIF) receptor 3 (LIFRO) and/or glycoprotein 130 (gp130), and a mitogen and trophic factor that promotes astrocyte survival and proliferation, as well as a resting/quiescent astrocyte state.

In some embodiments, the culture media is an astrocyte activation media capable of promoting the transition of astrocytes to a damaging reactive state. In some embodiments, the astrocyte activation culture medium comprises a serum-free basal medium, an antioxidant, an iron carrier that regulates iron homeostasis, a polyamine that promotes cell division, a hormone that activates the progesterone receptor, and one or more microglia-derived reactive astrocyte drivers (or proinflammatory cytokines) that promote the transition of astrocytes to a damaging reactive state.

In some embodiments, the serum-free basal medium comprises a basal medium (1) devoid of proteins, lipids, or growth factors that contains sufficient glucose, amino acids, vitamins, an inorganic salt to support the growth of mammalian cells, (2) comprises sufficient energy source (such as glucose), nitrogen source, carbon source, amino acids, vitamins, an inorganic salts to support growth of mammalian neuronal cells in the absence of feeder cells. Optionally, the serum-free basal medium further comprises (3) an amino acid supplement as a source of L-glutamine (such as GLUTAMAX™ brand of L-alanyl-L-glutamine dipeptide); optionally about 1-3 μM (e.g., about 2 mM μM) L-alanyl-L-glutamine dipeptide, and/or (4) a source of sodium pyruvate; optionally, about 0.5-1.5 mM (e.g., about 1 mM) sodium pyruvate. In some embodiments, the basal medium is DMEM and/or Neurobasal media, such as a 1:1 mixture of DMEM/Neurobasal medium.

In some embodiments, the basal medium comprises an amino acid supplement. In some embodiments, the amino acid supplement serves as an auxiliary energy source for rapidly dividing cells, such as, but not limited to, L-glutamine (GLUTAMAX™). In some embodiments, the amino acid supplement is at a concentration of at least 0.5 μM, at least 0.8 μM, at least 1.0 μM, at least 1.2 μM, at least 1.5 μM, at least 1.8 μM, at least 2.0 μM, at least 2.2 μM, at least 2.5 μM, at least 2.8 μM, at least 3.0 μM, at least 3.2 μM, or at least 3.5 μM. In some embodiments, the amino acid supplement is at a concentration of between about 0.5 μM and about 3.5 μM, between about 0.5 μM and about 3.2 μM, between about 0.5 μM and about 3.0 μM, between about 0.5 μM and about 2.8 μM, between about 0.5 μM and about 2.5 μM, between about 0.5 μM and about 2.2 μM, between about 0.5 μM and about 2.0 μM, between about 0.5 μM and about 1.8 μM, between about 0.5 μM and about 1.5 μM, between about 0.5 μM and about 1.2 μM, between about 0.5 μM and about 1.0 μM, between about 0.5 μM and about 0.8 μM, 0.8 μM and about 3.5 μM, between about 0.8 μM and about 3.2 μM, between about 0.8 μM and about 3.0 μM, between about 0.8 μM and about 2.8 μM, between about 0.8 μM and about 2.5 μM, between about 0.8 μM and about 2.2 μM, between about 0.8 μM and about 2.0 μM, between about 0.8 μM and about 1.8 μM, between about 0.8 μM and about 1.5 μM, between about 0.8 μM and about 1.2 μM, between about 0.8 μM and about 1.0 μM, between about 0.8 μM and about 0.8 μM, 1.0 μM and about 3.5 μM, between about 1.0 μM and about 3.2 μM, between about 1.0 μM and about 1.0 μM, between about 1.0 μM and about 2.8 μM, between about 1.0 μM and about 2.5 μM, between about 1.0 μM and about 2.2 μM, between about 1.0 μM and about 2.0 μM, between about 1.0 μM and about 1.8 μM, between about 1.0 μM and about 1.5 μM, between about 1.0 μM and about 1.2 μM, 1.2 μM and about 3.5 μM, between about 1.2 μM and about 3.2 μM, between about 1.2 μM and about 3.0 μM, between about 1.2 μM and about 2.8 μM, between about 1.2 μM and about 2.5 μM, between about 1.2 μM and about 2.2 μM, between about 1.2 μM and about 2.0 μM, between about 1.2 μM and about 1.8 μM, between about 1.2 μM and about 1.5 μM, 1.5 μM and about 3.5 μM, between about 1.5 μM and about 3.2 μM, between about 1.5 μM and about 3.0 μM, between about 1.5 μM and about 2.8 μM, between about 1.5 μM and about 2.5 μM, between about 1.5 μM and about 2.2 μM, between about 1.5 μM and about 2.0 μM, between about 1.5 μM and about 1.8 μM, 1.8 μM and about 3.5 μM, between about 1.8 μM and about 3.2 μM, between about 1.8 μM and about 3.0 μM, between about 1.8 μM and about 2.8 μM, between about 1.8 μM and about 2.5 μM, between about 1.8 μM and about 2.2 μM, between about 1.8 μM and about 2.0 μM, 2.0 μM and about 3.5 μM, between about 2.0 μM and about 3.2 μM, between about 2.0 μM and about 3.0 μM, between about 2.0 μM and about 2.8 μM, between about 2.0 μM and about 2.5 μM, between about 2.0 μM and about 2.2 μM, 2.2 μM and about 3.5 μM, between about 2.2 μM and about 3.2 μM, between about 2.2 μM and about 3.0 μM, between about 2.2 μM and about 2.8 μM, between about 2.2 μM and about 2.5 μM, 2.5 μM and about 3.5 μM, between about 2.5 μM and about 3.2 μM, between about 2.5 μM and about 3.0 μM, between about 2.5 μM and about 2.8 μM, 2.8 μM and about 3.5 μM, between about 2.8 μM and about 3.2 μM, between about 2.8 μM and about 3.0 μM, 3.0 μM and about 3.5 μM, between about 3.0 μM and about 3.2 μM, or 3.2 μM and about 3.5 μM.

In some embodiments, the basal medium comprises a carbon source, such as, but not limited to, sodium pyruvate. In some embodiments, the sodium pyruvate at a concentration of at least 0.5 mM, at least 0.8 mM, at least 1.0 mM, at least 1.2 mM, at least 1.5 mM, at least 1.8 mM, or at least 2.0 mM. In some embodiments, the carbon source at a concentration between about 0.5 mM and about 2.0 mM, between about 0.5 mM and about 1.8 mM, between about 0.5 mM and about 1.5 mM, between about 0.5 mM and about 1.2 mM, between about 0.5 mM and about 1.0 mM, between about 0.5 mM and about 0.8 mM, between about 0.8 mM and about 2.0 mM, between about 0.8 mM and about 1.8 mM, between about 0.8 mM and about 1.5 mM, between about 0.8 mM and about 1.2 mM, between about 0.8 mM and about 1.0 mM, between about 1.0 mM and about 2.0 mM, between about 1.0 mM and about 1.8 mM, between about 1.0 mM and about 1.5 mM, between about 1.0 mM and about 1.2 mM, between about 1.2 mM and about 2.0 mM, between about 1.2 mM and about 1.8 mM, between about 1.2 mM and about 1.5 mM, between about 1.5 mM and about 2.0 mM, between about 1.5 mM and about 1.8 mM, or between about 1.8 mM and about 2.0 mM.

In some embodiments, the resting astrocyte culture medium, astrocyte enrichment and/or maturation culture medium, and/or the astrocyte activation culture medium comprises an antioxidant. In some embodiments, the antioxidant is sodium selenite and N-acetylcysteine (NAC), catalase, reduced glutathione, alpha-tocopherol, or superoxide dismutase. In some embodiments, the antioxidant is NAC and is at a concentration of at least 15 μM, at least 20 μM, at least 25 μM, at least 30 μM, at least 35 μM, at least 40 μM, or at least 45 μM. In some embodiments, the concentration of NAC is between about 15 μM and about 45 μM, between about 15 μM and about 40 μM, between about 15 μM and about 35 μM, between about 15 μM and about 30 μM, between about 15 μM and about 25 μM, between about 15 μM and about 20 μM, between about 20 μM and about 45 μM, between about 20 μM and about 40 μM, between about 20 μM and about 35 μM, between about 20 μM and about 30 μM, between about 20 μM and about 25 μM, between about 25 μM and about 45 μM, between about 25 μM and about 40 μM, between about 25 μM and about 35 μM, between about 25 μM and about 30 μM, between about 30 μM and about 45 μM, between about 30 μM and about 40 μM, between about 30 μM and about 35 μM, between about 35 μM and about 45 μM, between about 35 μM and about 40 μM, or between about 40 μM and about 45 μM. In some embodiments, the concentration of NAC is about 30 μM.

In some embodiments, the resting astrocyte culture medium, astrocyte enrichment and/or maturation culture medium, and/or the astrocyte activation culture medium comprises a supplemental reagent combination of a growth factor and helps cells utilize glucose and amino acids (e.g., insulin), an iron carrier that regulates iron homeostasis in the cells (e.g., transferrin), an antioxidant that inhibits lipid peroxidation (e.g., sodium selenite), a polyamine necessary for proper cell division (e.g., putrescine), and/or a hormone that activates the progesterone receptor that promotes cell growth (e.g., progesterone). In some embodiments, the supplemental reagent combination is N2 Max Supplement.

In some embodiments, the growth factor and helps cells utilize glucose and amino acids is insulin or insulin like growth factor 1 (IGF-1). In some embodiments, the insulin or IGF-1 is not included. In some embodiments, the insulin is at a concentration of at least about 0.1 μg/mL, at least 0.5 μg/mL, at least 1.0 μg/mL, at least 1.5 μg/mL, at least 2.0 μg/mL, at least 2.5 μg/mL, at least 3.0 μg/mL, at least 3.5 μg/mL, at least 4.0 μg/mL, at least 4.5 μg/mL, at least 5.0 μg/mL, at least 6.0 μg/mL, at least 7.0 μg/mL, at least 8.0 μg/mL, at least 9.0 μg/mL, at least 10.0 μg/mL, at least 11.0 μg/mL, at least 12.0 μg/mL, at least 13.0 μg/mL, at least 14.0 μg/mL, at least 15.0 μg/mL, at least 16.0 μg/mL, at least 17.0 μg/mL, at least 18.0 μg/mL, at least 19.0, at least 20.0 μg/mL, at least 21.0 μg/mL, at least 22.0 μg/mL, at least 23.0 μg/mL, at least 24.0 μg/mL, at least 25.0 μg/mL, or at least 30.0 μg/mL. In some embodiments, the insulin or IGF-1 is at a concentration between about 0.1 μg/mL and about 30.0 μg/mL, between 0.1 μg/mL and about 25.0 μg/mL, between about 0.1 μg/mL and about 20.0 μg/mL, between about 0.1 μg/mL and about 15.0 μg/mL, between about 0.1 μg/mL and about 10.0 μg/mL, between about 0.1 μg/mL and about 5.0 μg/mL, between about 0.1 μg/mL and about 1.0 μg/mL, between about 1.0 μg/mL and about 30.0 μg/mL, between 1.0 μg/mL and about 25.0 μg/mL, between about 1.0 μg/mL and about 20.0 μg/mL, between about 1.0 μg/mL and about 15.0 μg/mL, between about 1.0 μg/mL and about 10.0 μg/mL, between about 1.0 μg/mL and about 5.0 μg/mL, between about 5.0 μg/mL and about 30.0 μg/mL, between 5.0 μg/mL and about 25.0 μg/mL, between about 5.0 μg/mL and about 20.0 μg/mL, between about 5.0 μg/mL and about 15.0 μg/mL, between about 5.0 μg/mL and about 10.0 μg/mL, between about 10.0 μg/mL and about 30.0 μg/mL, between 10.0 μg/mL and about 25.0 μg/mL, between about 10.0 μg/mL and about 20.0 μg/mL, between about 10.0 μg/mL and about 15.0 μg/mL, between about 15.0 μg/mL and about 30.0 μg/mL, between 15.0 μg/mL and about 25.0 μg/mL, between about 15.0 μg/mL and about 20.0 μg/mL, between about 20.0 μg/mL and about 30.0 μg/mL, between 20.0 μg/mL and about 25.0 μg/mL, or between about 25.0 μg/mL and about 30.0 μg/mL. In some embodiments, the insulin or IGF-1 is at a concentration between 0 μg/mL and about 25 μg/mL.

In some embodiments, the iron carrier that regulates iron homeostasis in the cells is transferrin. In some embodiments, the concentration of transferrin is at least about 50 μg/mL, at least about 100 μg/mL, or at least about 150 μg/mL. In some embodiments, the concentration of transferrin is between about 50 μg/mL and about 150 μg/mL, between about 50 μg/mL and about 120 μg/mL, between about 50 μg/mL and about 100 μg/mL, between about 50 μg/mL and about 75 μg/mL, between about 75 μg/mL and about 150 μg/mL, between about 75 μg/mL and about 120 μg/mL, between about 75 μg/mL and about 100 μg/mL, between about 100 μg/mL and about 150 μg/mL, between about 100 μg/mL and about 120 μg/mL, or between about 120 μg/mL and about 150 μg/mL. In some embodiments, the transferrin is at a concentration of 100 μg/mL.

In some embodiments, the antioxidant that inhibits lipid peroxidation is sodium selenite. In some embodiments, the sodium selenite is at a concentration between about 5.2 ng/mL to about 40 ng/mL. In some embodiments, the concentration of sodium selenite is at least 1.0 ng/mL, at least 2.0 ng/mL, at least 3.0 ng/mL, at least 4.0 ng/mL, at least 5.0 ng/mL, at least 5.2 ng/mL, at least 5.5 ng/mL, at least 5.8 ng/mL, at least 6.0 ng/mL, at least 7.0 ng/mL, at least 8.0 ng/mL, at least 9.0 ng/mL, at least 10.0 ng/mL, at least 15 ng/mL, at least 20 ng/mL, at least 25 ng/mL, at least 30 ng/mL, at least 35 ng/mL, at least 40 ng/mL, or at least 45 ng/mL. In some embodiments, the sodium selenite is at a concentration between about 5 ng/mL and about 45 ng/mL, between about 5 ng/mL and about 40 ng/mL, between about 5 ng/mL and about 35 ng/mL, between about 5 ng/mL and about 30 ng/mL, between about 5 ng/mL and about 25 ng/mL, between about 5 ng/mL and about 20 ng/mL, between about 5 ng/mL and about 15 ng/mL, between about 5 ng/mL and about 10 ng/mL, between about 10 ng/mL and about 45 ng/mL, between about 10 ng/mL and about 40 ng/mL, between about 10 ng/mL and about 35 ng/mL, between about 10 ng/mL and about 30 ng/mL, between about 10 ng/mL and about 25 ng/mL, between about 10 ng/mL and about 20 ng/mL, between about 10 ng/mL and about 15 ng/mL, between about 15 ng/mL and about 45 ng/mL, between about 15 ng/mL and about 40 ng/mL, between about 15 ng/mL and about 35 ng/mL, between about 15 ng/mL and about 30 ng/mL, between about 15 ng/mL and about 25 ng/mL, between about 15 ng/mL and about 20 ng/mL, between about 20 ng/mL and about 45 ng/mL, between about 20 ng/mL and about 40 ng/mL, between about 20 ng/mL and about 35 ng/mL, between about 20 ng/mL and about 30 ng/mL, between about 20 ng/mL and about 25 ng/mL, between about 25 ng/mL and about 45 ng/mL, between about 25 ng/mL and about 40 ng/mL, between about 25 ng/mL and about 35 ng/mL, between about 25 ng/mL and about 30 ng/mL, between about 30 ng/mL and about 45 ng/mL, between about 30 ng/mL and about 40 ng/mL, between about 30 ng/mL and about 35 ng/mL, between about 35 ng/mL and about 45 ng/mL, between about 35 ng/mL and about 40 ng/mL, or between about 40 ng/mL and about 45 ng/mL. In some embodiments, the sodium selenite is at a concentration between about 10 ng/mL and about 20 ng/mL.

In some embodiments, the a polyamine mitogen that promotes cell division is putrescine. In some embodiments, the putrescine is at a concentration of at least 5 μg/mL, at least 6 μg/mL, at least 7 μg/mL, at least 8 μg/mL, at least 9 μg/mL, at least 10 μg/mL, at least 12 μg/mL, at least 14 μg/mL, at least 16 μg/mL, at least 18 μg/mL, at least 20 μg/mL, at least 22 μg/mL, at least 24 μg/mL, at least 26 μg/mL, at least 28 μg/mL, at least 30 μg/mL, at least 32 μg/mL, or at least 34 μg/mL. In some embodiments, the putrescine is at a concentration between about 5 μg/mL and about 34 μg/mL, between about 5 μg/mL and about 32 μg/mL, between about 5 μg/mL and about 30 μg/mL, between about 5 μg/mL and about 28 μg/mL, between about 5 μg/mL and about 26 μg/mL, between about 5 μg/mL and about 24 μg/mL, between about 5 μg/mL and about 22 μg/mL, between about 5 μg/mL and about 20 μg/mL, between about 5 μg/mL and about 18 μg/mL, between about 5 μg/mL and about 16 μg/mL, between about 5 μg/mL and about 14 μg/mL, between about 5 μg/mL and about 12 μg/mL, between about 5 μg/mL and about 10 μg/mL, between about 5 μg/mL and about 8 μg/mL, between about 8 μg/mL and about 34 μg/mL, between about 8 μg/mL and about 32 μg/mL, between about 8 μg/mL and about 30 μg/mL, between about 8 μg/mL and about 28 μg/mL, between about 8 μg/mL and about 26 μg/mL, between about 8 μg/mL and about 24 μg/mL, between about 8 μg/mL and about 22 μg/mL, between about 8 μg/mL and about 20 μg/mL, between about 8 μg/mL and about 18 μg/mL, between about 8 μg/mL and about 16 μg/mL, between about 8 μg/mL and about 14 μg/mL, between about 8 μg/mL and about 12 μg/mL, between about 8 μg/mL and about 10 μg/mL, between about 10 μg/mL and about 34 μg/mL, between about 10 μg/mL and about 32 μg/mL, between about 10 μg/mL and about 30 μg/mL, between about 10 μg/mL and about 28 μg/mL, between about 10 μg/mL and about 26 μg/mL, between about 10 μg/mL and about 24 μg/mL, between about 10 μg/mL and about 22 μg/mL, between about 10 μg/mL and about 20 μg/mL, between about 10 μg/mL and about 18 μg/mL, between about 10 μg/mL and about 16 μg/mL, between about 10 μg/mL and about 14 μg/mL, between about 10 μg/mL and about 12 μg/mL, between about 12 μg/mL and about 34 μg/mL, between about 12 μg/mL and about 32 μg/mL, between about 12 μg/mL and about 30 μg/mL, between about 12 μg/mL and about 28 μg/mL, between about 12 μg/mL and about 26 μg/mL, between about 12 μg/mL and about 24 μg/mL, between about 12 μg/mL and about 22 μg/mL, between about 12 μg/mL and about 20 μg/mL, between about 12 μg/mL and about 18 μg/mL, between about 12 μg/mL and about 16 μg/mL, between about 12 μg/mL and about 14 μg/mL, between about 14 μg/mL and about 34 μg/mL, between about 14 μg/mL and about 32 μg/mL, between about 14 μg/mL and about 30 μg/mL, between about 14 μg/mL and about 28 μg/mL, between about 14 μg/mL and about 26 μg/mL, between about 14 μg/mL and about 24 μg/mL, between about 14 μg/mL and about 22 μg/mL, between about 14 μg/mL and about 20 μg/mL, between about 14 μg/mL and about 18 μg/mL, between about 14 μg/mL and about 16 μg/mL, between about 16 μg/mL and about 34 μg/mL, between about 16 μg/mL and about 32 μg/mL, between about 16 μg/mL and about 30 μg/mL, between about 16 μg/mL and about 28 μg/mL, between about 16 μg/mL and about 26 μg/mL, between about 16 μg/mL and about 24 μg/mL, between about 16 μg/mL and about 22 μg/mL, between about 16 μg/mL and about 20 μg/mL, between about 16 μg/mL and about 18 μg/mL, between about 18 μg/mL and about 34 μg/mL, between about 18 μg/mL and about 32 μg/mL, between about 18 μg/mL and about 30 μg/mL, between about 18 μg/mL and about 28 μg/mL, between about 18 μg/mL and about 26 μg/mL, between about 18 μg/mL and about 24 μg/mL, between about 18 μg/mL and about 22 μg/mL, between about 18 μg/mL and about 20 μg/mL, between about 20 μg/mL and about 34 μg/mL, between about 20 μg/mL and about 32 μg/mL, between about 20 μg/mL and about 30 μg/mL, between about 20 μg/mL and about 28 μg/mL, between about 20 μg/mL and about 26 μg/mL, between about 20 μg/mL and about 24 μg/mL, between about 20 μg/mL and about 22 μg/mL, between about 22 μg/mL and about 34 μg/mL, between about 22 μg/mL and about 32 μg/mL, between about 22 μg/mL and about 30 μg/mL, between about 22 μg/mL and about 28 μg/mL, between about 22 μg/mL and about 26 μg/mL, between about 22 μg/mL and about 24 μg/mL, between about 24 μg/mL and about 34 μg/mL, between about 24 μg/mL and about 32 μg/mL, between about 24 μg/mL and about 30 μg/mL, between about 24 μg/mL and about 28 μg/mL, between about 24 μg/mL and about 26 μg/mL, between about 26 μg/mL and about 34 μg/mL, between about 26 μg/mL and about 32 μg/mL, between about 26 μg/mL and about 30 μg/mL, between about 26 μg/mL and about 28 μg/mL, between about 28 μg/mL and about 34 μg/mL, between about 28 μg/mL and about 32 μg/mL, between about 28 μg/mL and about 30 μg/mL, between about 30 μg/mL and about 34 μg/mL, between about 30 μg/mL and about 32 μg/mL, or 32 μg/mL and about 34 μg/mL. In some embodiments, the putrescine is at a concentration at about 16 μg/mL.

In some embodiments, the hormone that activates the progesterone receptor that promotes cell growth is progesterone. In some embodiments, the progesterone is at a concentration of at least 5 ng/mL, at least 6 ng/mL, at least 10 ng/mL, at least 15 ng/mL, at least 20, at least 25 ng/mL, at least 30 ng/mL, at least 35 ng/mL, at least 40 ng/mL, at least 45 ng/mL, at least 50 ng/mL, at least 55 ng/mL, at least 60 ng/mL, or at least 65 ng/mL. In some embodiments, the progesterone is at a concentration between about 5 ng/mL and about 65 ng/mL, between about 5 ng/mL and about 60 ng/mL, between about 5 ng/mL and about 55 ng/mL, between about 5 ng/mL and about 50 ng/mL, between about 5 ng/mL and about 45 ng/mL, between about 5 ng/mL and about 40 ng/mL, between about 5 ng/mL and about 35 ng/mL, between about 5 ng/mL and about 30 ng/mL, between about 5 ng/mL and about 25 ng/mL, between about 5 ng/mL and about 20 ng/mL, between about 5 ng/mL and about 15 ng/mL, between about 5 ng/mL and about 10 ng/mL, between about 10 ng/mL and about 65 ng/mL, between about 10 ng/mL and about 60 ng/mL, between about 10 ng/mL and about 55 ng/mL, between about 10 ng/mL and about 50 ng/mL, between about 10 ng/mL and about 45 ng/mL, between about 10 ng/mL and about 40 ng/mL, between about 10 ng/mL and about 35 ng/mL, between about 10 ng/mL and about 30 ng/mL, between about 10 ng/mL and about 25 ng/mL, between about 10 ng/mL and about 20 ng/mL, between about 10 ng/mL and about 15 ng/mL, between about 15 ng/mL and about 65 ng/mL, between about 15 ng/mL and about 60 ng/mL, between about 15 ng/mL and about 55 ng/mL, between about 15 ng/mL and about 50 ng/mL, between about 15 ng/mL and about 45 ng/mL, between about 15 ng/mL and about 40 ng/mL, between about 15 ng/mL and about 35 ng/mL, between about 15 ng/mL and about 30 ng/mL, between about 15 ng/mL and about 25 ng/mL, between about 15 ng/mL and about 20 ng/mL, between about 20 ng/mL and about 65 ng/mL, between about 20 ng/mL and about 60 ng/mL, between about 20 ng/mL and about 55 ng/mL, between about 20 ng/mL and about 50 ng/mL, between about 20 ng/mL and about 45 ng/mL, between about 20 ng/mL and about 40 ng/mL, between about 20 ng/mL and about 35 ng/mL, between about 20 ng/mL and about 30 ng/mL, between about 20 ng/mL and about 25 ng/mL, between about 25 ng/mL and about 65 ng/mL, between about 25 ng/mL and about 60 ng/mL, between about 25 ng/mL and about 55 ng/mL, between about 25 ng/mL and about 50 ng/mL, between about 25 ng/mL and about 45 ng/mL, between about 25 ng/mL and about 40 ng/mL, between about 25 ng/mL and about 35 ng/mL, between about 25 ng/mL and about 30 ng/mL, between about 30 ng/mL and about 65 ng/mL, between about 30 ng/mL and about 60 ng/mL, between about 30 ng/mL and about 55 ng/mL, between about 30 ng/mL and about 50 ng/mL, between about 30 ng/mL and about 45 ng/mL, between about 30 ng/mL and about 40 ng/mL, between about 30 ng/mL and about 35 ng/mL, between about 35 ng/mL and about 65 ng/mL, between about 35 ng/mL and about 60 ng/mL, between about 35 ng/mL and about 55 ng/mL, between about 35 ng/mL and about 50 ng/mL, between about 35 ng/mL and about 45 ng/mL, between about 35 ng/mL and about 40 ng/mL, between about 40 ng/mL and about 65 ng/mL, between about 40 ng/mL and about 60 ng/mL, between about 40 ng/mL and about 55 ng/mL, between about 40 ng/mL and about 50 ng/mL, between about 40 ng/mL and about 45 ng/mL, between about 45 ng/mL and about 65 ng/mL, between about 45 ng/mL and about 60 ng/mL, between about 45 ng/mL and about 55 ng/mL, between about 45 ng/mL and about 50 ng/mL, between about 50 ng/mL and about 65 ng/mL, between about 50 ng/mL and about 60 ng/mL, between about 50 ng/mL and about 55 ng/mL, between about 55 ng/mL and about 65 ng/mL, between about 55 ng/mL and about 60 ng/mL, or between about 60 ng/mL and about 65 ng/mL. In some embodiments, the progesterone is at a concentration of 30 ng/mL. In some embodiments, the progesterone is at a concentration of about 6 ng/mL to about 60 ng/mL.

In some embodiments, the astrocyte enrichment medium and/or maturation culture medium comprises an IL-6 superfamily cytokine that actives STAT signaling through leukemia inhibitory factor (LIF) receptor R (LIFRO) and/or glycoprotein 130 (gp130). In some embodiments, the IL-6 superfamily cytokine is ciliary neurotrophic factor (CNTF). In some embodiments, the concentration of CNTF is about 5 ng/mL to about 20 ng/mL. In some embodiments, the concentration of CNTF is about 10 ng/mL. In some embodiments, the concentration of CNTF is at least 5 ng/mL, at least 10 ng/mL, at least 15 ng/mL, at least 20 ng/mL, or at least 25 ng/mL. In some embodiments, the concentration of CNTF is between about 5 ng/mL and about 25 ng/mL, between about 5 ng/mL and about 20 ng/mL, between about 5 ng/mL and about 15 ng/mL, between about 5 ng/mL and about 10 ng/mL, between about 10 ng/mL and about 25 ng/mL, between about 10 ng/mL and about 20 ng/mL, between about 10 ng/mL and about 15 ng/mL, between about 15 ng/mL and about 25 ng/mL, between about 15 ng/mL and about 20 ng/mL, or between about 20 ng/mL and about 25 ng/mL.

In some embodiments, the astrocyte enrichment medium and/or maturation culture medium comprises a TGF-beta superfamily cytokine that promotes astrogenesis and astrocyte maturation through SMAD dependent-signaling. In some embodiments, the TGF-beta superfamily cytokine that promotes astrogenesis and astrocyte maturation through SMAD dependent-signaling is a transforming growth factor R (TGF-P) family member. In some embodiments, the TGF-P family member is a bone morphogenetic protein (BMP). In some embodiments, the BMP is BMP2, BMP4, BMP5, BMP6, BMP7, BMP10 or BMP15.

In some embodiments, the concentration of BMP is about 5 ng/mL to about 100 ng/mL. In some embodiments, the concentration of BMP is at least 5 ng/mL, at least 10 ng/mL, at least 15 ng/mL, at least 20 ng/mL, at least 25 ng/mL, at least 30 ng/mL, at least 35 ng/mL, at least 40 ng/mL, at least 45 ng/mL at least 50 ng/mL, at least 55 ng/mL, at least 60 ng/mL, at least 65 ng/mL, at least 70 ng/mL, at least 75 ng/mL, at least 80 ng/mL, at least 85 ng/mL, at least 90 ng/mL, at least 95 ng/mL, or at least 100 ng/mL. In some embodiments, the concentration of BMP is between about 5 ng/mL and about 100 ng/mL, between about 5 ng/mL and about 90 ng/mL, between about 5 ng/mL and about 80 ng/mL, between about 5 ng/mL and about 70 ng/mL, between about 5 ng/mL and about 60 ng/mL, between about 5 ng/mL and about 50 ng/mL, between about 5 ng/mL and about 40 ng/mL, between about 5 ng/mL and about 30 ng/mL, between about 5 ng/mL and about 20 ng/mL, between about 5 ng/mL and about 10 ng/mL, between about 10 ng/mL and about 100 ng/mL, between about 10 ng/mL and about 90 ng/mL, between about 10 ng/mL and about 80 ng/mL, between about 10 ng/mL and about 70 ng/mL, between about 10 ng/mL and about 60 ng/mL, between about 10 ng/mL and about 50 ng/mL, between about 10 ng/mL and about 40 ng/mL, between about 10 ng/mL and about 30 ng/mL, between about 10 ng/mL and about 20 ng/mL, between about 20 ng/mL and about 100 ng/mL, between about 20 ng/mL and about 90 ng/mL, between about 20 ng/mL and about 80 ng/mL, between about 20 ng/mL and about 70 ng/mL, between about 20 ng/mL and about 60 ng/mL, between about 20 ng/mL and about 50 ng/mL, between about 20 ng/mL and about 40 ng/mL, between about 20 ng/mL and about 30 ng/mL, between about 30 ng/mL and about 100 ng/mL, between about 30 ng/mL and about 90 ng/mL, between about 30 ng/mL and about 80 ng/mL, between about 30 ng/mL and about 70 ng/mL, between about 30 ng/mL and about 60 ng/mL, between about 30 ng/mL and about 50 ng/mL, between about 30 ng/mL and about 40 ng/mL, between about 40 ng/mL and about 100 ng/mL, between about 40 ng/mL and about 90 ng/mL, between about 40 ng/mL and about 80 ng/mL, between about 40 ng/mL and about 70 ng/mL, between about 40 ng/mL and about 60 ng/mL, between about 40 ng/mL and about 50 ng/mL, between about 50 ng/mL and about 100 ng/mL, between about 50 ng/mL and about 90 ng/mL, between about 50 ng/mL and about 80 ng/mL, between about 50 ng/mL and about 70 ng/mL, between about 50 ng/mL and about 60 ng/mL, between about 60 ng/mL and about 100 ng/mL, between about 60 ng/mL and about 90 ng/mL, between about 60 ng/mL and about 80 ng/mL, between about 60 ng/mL and about 70 ng/mL, between about 70 ng/mL and about 100 ng/mL, between about 70 ng/mL and about 80 ng/mL, between about 70 ng/mL and about 90 ng/mL, between about 80 ng/mL and about 100 ng/mL, between about 80 ng/mL and about 90 ng/mL, or between about 90 ng/mL and about 100 ng/mL. In some embodiments, the concentration of BMP is between about 5 ng/mL to about 20 ng/mL.

In some embodiments, the resting astrocyte culture medium and/or astrocyte enrichment medium and/or maturation culture medium comprises a tropic factor, e.g., neurotrophic factor. In some embodiments, the neurotrophic factor is heparin binding EGF like growth factor (HBEGF). In some embodiments, the concentration of HBEGF is about 5 ng/mL to about 10 ng/mL. In some embodiments, the concentration of HBEGF is at least 2 ng/mL, at least 3 ng/mL, at least 4 ng/mL, at least 5 ng/mL, at least 6 ng/mL, at least 7 ng/mL, at least 8 ng/mL, at least 9 ng/mL, or at least 10 ng/mL. In some embodiments, the concentration of HBEGF is between about 2 ng/mL to about 10 ng/mL, between about 2 ng/mL to about 9 ng/mL, between about 2 ng/mL to about 8 ng/mL, between about 2 ng/mL to about 7 ng/mL, between about 2 ng/mL to about 6 ng/mL, between about 2 ng/mL to about 5 ng/mL, between about 2 ng/mL to about 4 ng/mL, between about 2 ng/mL to about 3 ng/mL, between about 3 ng/mL to about 10 ng/mL, between about 3 ng/mL to about 9 ng/mL, between about 3 ng/mL to about 8 ng/mL, between about 3 ng/mL to about 7 ng/mL, between about 3 ng/mL to about 6 ng/mL, between about 3 ng/mL to about 5 ng/mL, between about 3 ng/mL to about 4 ng/mL, between about 4 ng/mL to about 10 ng/mL, between about 4 ng/mL to about 9 ng/mL, between about 4 ng/mL to about 8 ng/mL, between about 4 ng/mL to about 7 ng/mL, between about 4 ng/mL to about 6 ng/mL, between about 4 ng/mL to about 5 ng/mL, between about 5 ng/mL to about 10 ng/mL, between about 5 ng/mL to about 9 ng/mL, between about 5 ng/mL to about 8 ng/mL, between about 5 ng/mL to about 7 ng/mL, between about 5 ng/mL to about 6 ng/mL, between about 6 ng/mL to about 10 ng/mL, between about 6 ng/mL to about 9 ng/mL, between about 6 ng/mL to about 8 ng/mL, between about 6 ng/mL to about 7 ng/mL, between about 7 ng/mL to about 10 ng/mL, between about 7 ng/mL to about 9 ng/mL, between about 7 ng/mL to about 8 ng/mL, between about 8 ng/mL to about 10 ng/mL, between about 8 ng/mL to about 9 ng/mL, or between about 9 ng/mL to about 10 ng/mL. In some embodiments, the concentration of HBEGF is about 5 ng/mL.

In some embodiments, the astrocyte enrichment medium and/or maturation culture medium comprises a mitogen and trophic factor that promotes astrocyte survival and proliferation, as well as a resting/quiescent astrocyte state. In some embodiments, the mitogen and trophic factor is fibroblast growth factor 2 (FGF2). In some embodiments, the concentration of FGF2 is between about 10 ng/mL and about 40 ng/mL. In some embodiments, the concentration of FGF2 is about 20 ng/mL. In some embodiments, the concentration of FGF2 is at least 10 ng/mL, at least 15 ng/mL, at least 20 ng/mL, at least 25 ng/mL, at least 30 ng/mL, at least 35, or at least 40 ng/mL. In some embodiments, the concentrations of FGF2 is between about 10 ng/mL to about 40 ng/mL, between about 10 ng/mL to about 35 ng/mL, between about 10 ng/mL to about 30 ng/mL, between about 10 ng/mL to about 25 ng/mL, between about 10 ng/mL to about 20 ng/mL, between about 10 ng/mL to about 15 ng/mL, between about 15 ng/mL to about 40 ng/mL, between about 15 ng/mL to about 35 ng/mL, between about 15 ng/mL to about 30 ng/mL, between about 15 ng/mL to about 25 ng/mL, between about 15 ng/mL to about 20 ng/mL, between about 20 ng/mL to about 40 ng/mL, between about 20 ng/mL to about 35 ng/mL, between about 20 ng/mL to about 30 ng/mL, between about 20 ng/mL to about 25 ng/mL, between about 25 ng/mL to about 40 ng/mL, between about 25 ng/mL to about 35 ng/mL, between about 25 ng/mL to about 30 ng/mL, between about 30 ng/mL to about 40 ng/mL, between about 30 ng/mL to about 35 ng/mL, or between about 35 ng/mL to about 40 ng/mL.

In some embodiments, the astrocyte activation culture medium comprises reactive astrocyte drivers. In some embodiments, the reactive astrocyte drivers are TNFα, IL1α, and/or C1q.

In some embodiments, the concentration of IL-1α is between about 1 ng/mL to about 5 ng/mL. In some embodiments, the concentration of IL-1α is about 3 ng/mL. In some embodiments, the concentration of IL-1α is at least 1 ng/mL, at least 2 ng/mL, at least 3 ng/mL, at least 4 ng/mL, or at least 5 ng/mL. In some embodiments, the concentration of IL-1α is between about 1 ng/mL to about 5 ng/mL, between about 1 ng/mL to about 4 ng/mL, between about 1 ng/mL to about 3 ng/mL, between about 1 ng/mL to about 2 ng/mL, between about 2 ng/mL to about 5 ng/mL, between about 2 ng/mL to about 4 ng/mL, between about 2 ng/mL to about 3 ng/mL, between about 3 ng/mL to about 5 ng/mL, between about 3 ng/mL to about 4 ng/mL, or between about 4 ng/mL to about 5 ng/mL.

In some embodiments, the concentration of TNFα is between about 15 ng/mL and about 60 ng/mL. In some embodiments, the concentration of TNFα is about 30 ng/mL. In some embodiments, the concentration of TNFα is at least 15 ng/mL, at least 20 ng/mL, at least 25 ng/mL, at least 30 ng/mL, at least 35 ng/mL, at least 40 ng/mL, at least 45 ng/mL, at least 50 ng/mL, at least 55 ng/mL, or at least 60 ng/mL.

In some embodiments, the concentration of TNFα is between 15 ng/mL and about 60 ng/mL, between 15 ng/mL and about 55 ng/mL, between 15 ng/mL and about 50 ng/mL, between 15 ng/mL and about 45 ng/mL, between 15 ng/mL and about 40 ng/mL, between 15 ng/mL and about 35 ng/mL, between 15 ng/mL and about 30 ng/mL, between 15 ng/mL and about 25 ng/mL, between 15 ng/mL and about 20 ng/mL, between 20 ng/mL and about 60 ng/mL, between 20 ng/mL and about 55 ng/mL, between 20 ng/mL and about 50 ng/mL, between 20 ng/mL and about 45 ng/mL, between 20 ng/mL and about 40 ng/mL, between 20 ng/mL and about 35 ng/mL, between 20 ng/mL and about 30 ng/mL, between 20 ng/mL and about 25 ng/mL, between 25 ng/mL and about 60 ng/mL, between 25 ng/mL and about 55 ng/mL, between 25 ng/mL and about 50 ng/mL, between 25 ng/mL and about 45 ng/mL, between 25 ng/mL and about 40 ng/mL, between 25 ng/mL and about 35 ng/mL, between 25 ng/mL and about 30 ng/mL, between 30 ng/mL and about 60 ng/mL, between 30 ng/mL and about 55 ng/mL, between 30 ng/mL and about 50 ng/mL, between 30 ng/mL and about 45 ng/mL, between 30 ng/mL and about 40 ng/mL, between 30 ng/mL and about 35 ng/mL, between 35 ng/mL and about 60 ng/mL, between 35 ng/mL and about 55 ng/mL, between 35 ng/mL and about 50 ng/mL, between 35 ng/mL and about 45 ng/mL, between 35 ng/mL and about 40 ng/mL, between 40 ng/mL and about 60 ng/mL, between 40 ng/mL and about 55 ng/mL, between 40 ng/mL and about 50 ng/mL, between 40 ng/mL and about 45 ng/mL, between 45 ng/mL and about 60 ng/mL, between 45 ng/mL and about 55 ng/mL, between 45 ng/mL and about 50 ng/mL, between 50 ng/mL and about 60 ng/mL, between 50 ng/mL and about 55 ng/mL, or between 55 ng/mL and about 60 ng/mL.

In some embodiments, the concentration of C1q is between about 200 ng/mL and about 800 ng/mL. In some embodiments, the concentration of C1q is about 400 ng/mL. In some embodiments, the concentration of C1q is at least 200 ng/mL, at least 250 ng/mL, at least 300 ng/mL, at least 350 ng/mL, at least 400 ng/mL, at least 450 ng/mL, at least 450 ng/mL, at least 500 ng/mL, at least 550 ng/mL, at least 600 ng/mL, at least 650 ng/mL, at least 700 ng/mL, at least 750 ng/mL, or at least 800 ng/mL. In some embodiments, the concentration of C1q is between 200 ng/mL and about 800 ng/mL, between 200 ng/mL and about 750 ng/mL, between 200 ng/mL and about 700 ng/mL, between 200 ng/mL and about 650 ng/mL, between 200 ng/mL and about 600 ng/mL, between 200 ng/mL and about 550 ng/mL, between 200 ng/mL and about 500 ng/mL, between 200 ng/mL and about 450 ng/mL, between 200 ng/mL and about 400 ng/mL, between 200 ng/mL and about 350 ng/mL, between 200 ng/mL and about 300 ng/mL, between 200 ng/mL and about 250 ng/mL, between 250 ng/mL and about 800 ng/mL, between 250 ng/mL and about 750 ng/mL, between 250 ng/mL and about 700 ng/mL, between 250 ng/mL and about 650 ng/mL, between 250 ng/mL and about 600 ng/mL, between 250 ng/mL and about 550 ng/mL, between 250 ng/mL and about 500 ng/mL, between 250 ng/mL and about 450 ng/mL, between 250 ng/mL and about 400 ng/mL, between 250 ng/mL and about 350 ng/mL, between 250 ng/mL and about 300 ng/mL, between 300 ng/mL and about 800 ng/mL, between 300 ng/mL and about 750 ng/mL, between 300 ng/mL and about 700 ng/mL, between 300 ng/mL and about 650 ng/mL, between 300 ng/mL and about 600 ng/mL, between 300 ng/mL and about 550 ng/mL, between 300 ng/mL and about 500 ng/mL, between 300 ng/mL and about 450 ng/mL, between 300 ng/mL and about 400 ng/mL, between 300 ng/mL and about 350 ng/mL, between 350 ng/mL and about 800 ng/mL, between 350 ng/mL and about 750 ng/mL, between 350 ng/mL and about 700 ng/mL, between 350 ng/mL and about 650 ng/mL, between 350 ng/mL and about 600 ng/mL, between 350 ng/mL and about 550 ng/mL, between 350 ng/mL and about 500 ng/mL, between 350 ng/mL and about 450 ng/mL, between 350 ng/mL and about 400 ng/mL, between 400 ng/mL and about 800 ng/mL, between 400 ng/mL and about 750 ng/mL, between 400 ng/mL and about 700 ng/mL, between 400 ng/mL and about 650 ng/mL, between 400 ng/mL and about 600 ng/mL, between 400 ng/mL and about 550 ng/mL, between 400 ng/mL and about 500 ng/mL, between 400 ng/mL and about 450 ng/mL, between 450 ng/mL and about 800 ng/mL, between 450 ng/mL and about 750 ng/mL, between 450 ng/mL and about 700 ng/mL, between 450 ng/mL and about 650 ng/mL, between 450 ng/mL and about 600 ng/mL, between 450 ng/mL and about 550 ng/mL, between 450 ng/mL and about 500 ng/mL, between 500 ng/mL and about 800 ng/mL, between 500 ng/mL and about 750 ng/mL, between 500 ng/mL and about 700 ng/mL, between 500 ng/mL and about 650 ng/mL, between 500 ng/mL and about 600 ng/mL, between 500 ng/mL and about 550 ng/mL, between 550 ng/mL and about 800 ng/mL, between 550 ng/mL and about 750 ng/mL, between 550 ng/mL and about 700 ng/mL, between 550 ng/mL and about 650 ng/mL, between 550 ng/mL and about 600 ng/mL, between 600 ng/mL and about 800 ng/mL, between 600 ng/mL and about 750 ng/mL, between 600 ng/mL and about 700 ng/mL, between 600 ng/mL and about 650 ng/mL, between 650 ng/mL and about 800 ng/mL, between 650 ng/mL and about 750 ng/mL, between 650 ng/mL and about 700 ng/mL, between 700 ng/mL and about 800 ng/mL, between 700 ng/mL and about 750 ng/mL, or between 750 ng/mL and about 800 ng/mL.

In certain embodiments, resting astrocytes cultured in said resting astrocyte culture medium exhibit a stellate morphology, express canonical mature astrocyte markers (e.g., AQP4, GLT-1, VIMENTIN, GLAST, and/or ALDH1L1), and/or do not express reactive astrocyte markers (e.g., Lcn2, Steap4, and Cxc110).

In certain embodiments, said astrocyte enrichment and/or maturation culture medium promotes enrichment and/or maturation for astrocytes in astrocyte-containing neuronal tissues.

In certain embodiments, after culturing a mixture of astrocyte-containing cells in the astrocyte enrichment and/or maturation culture medium of the invention for a pre-determined period of time, such as to confluence with fresh media change once every two days, at least 70%, 75%, 80%, 85%, 90%, 95%, 96%, 97%, 98%, 99% or more of the surviving cells are astrocytes, such as astrocytes expressing canonical astrocyte markers.

In certain embodiments, the surviving astrocytes in the culture contain less than 10%, 8%, 5%, 2%, or 1% of non-astrocytes, such as oligodendrocytes, microglia, or neurons.

In certain embodiments, the surviving astrocytes in the culture substantially lack expression of oligodendrocyte markers, such as Sox10 or Mbp.

In certain embodiments, the surviving astrocytes in the culture substantially lack expression of neuronal markers, such as Snap25 or Nef1.

In certain embodiments, the surviving astrocytes in the culture substantially lack expression of microglia markers, such as Cd68 or Tmem119.

In certain embodiments, the resting astrocyte culture medium of various embodiments of the above aspects or any other aspect of the invention delineated herein comprises i) 1:1 mixture of DMEM/Neurobasal media mixture comprising sodium pyruvate and L-alanyl-L-glutamine dipeptide in the serum-free basal medium; ii) sodium selenite and NAC as said antioxidant; iii) transferrin as said iron carrier; iv) putrescine as said polyamine; v) progesterone as said hormone that activates the progesterone receptor; and, vi) HBEGF as said trophic factor that promotes astrocyte survival in culture.

In certain embodiments, the resting astrocyte culture medium of various embodiments of the above aspects or any other aspect of the invention delineated herein comprises i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture; ii) about 5.2-40 ng/mL (e.g., about 10-20 ng/mL) sodium selenite and about 20-40 μM (e.g., about 30 μM) NAC; iii) about 50-200 μg/mL (e.g., about 100 μg/mL) transferrin; iv) about 8-32 μg/mL (e.g., about 16 μg/mL) putrescine; v) about 6-60 ng/mL (e.g., about 30 ng/mL) progesterone; vi) about 0-25 μg/mL insulin; and, vii) about 5-10 ng/mL (e.g., about 5 ng/mL) HBEGF.

In certain specific embodiments, the resting astrocyte medium of the invention comprises: 1:1 DMEM/Neurobasal; 2 mM GLUTAMAX™; 1 mM Sodium Pyruvate; 30 μM NAC; 1×N2 Max; 5 ng/mL HBEGF.

In certain embodiments, the astrocyte enrichment and/or maturation culture medium of various embodiments of the above aspects or any other aspect of the invention delineated herein comprises: i) 1:1 mixture of DMEM/Neurobasal media mixture comprising sodium pyruvate and L-alanyl-L-glutamine dipeptide in the serum-free basal medium; ii) sodium selenite and NAC as said antioxidant; iii) transferrin as said iron carrier; iv) putrescine as said polyamine; v) progesterone as said hormone that activates the progesterone receptor; vi) HBEGF as said trophic factor that promotes astrocyte survival in culture; vii) BMP4 as the TGF-beta superfamily cytokine; viii) CNTF as the IL-6 superfamily cytokine; and, ix) FGF2 as the mitogen and trophic factor.

In certain embodiments, the astrocyte enrichment and/or maturation culture medium of various embodiments of the above aspects or any other aspect of the invention delineated herein comprises: i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture; ii) about 5.2-40 ng/mL (e.g., about 10-20 ng/mL) sodium selenite and about 20-40 μM (e.g., about 30 μM) NAC; iii) about 50-200 μg/mL (e.g., about 100 μg/mL) transferrin; iv) about 8-32 μg/mL (e.g., about 16 μg/mL) putrescine; v) about 6-60 ng/mL (e.g., about 30 ng/mL) progesterone; vi) about 0-25 μg/mL insulin; vii) about 2-10 ng/mL (e.g., about 5 ng/mL) HBEGF; viii) about 5-20 ng/mL (e.g., about 10 ng/mL) CNTF; ix)

about 10-40 ng/mL (about 20 ng/mL) FGF2; and, x) about 5-20 ng/mL (e.g., about 10 ng/mL) BMP4.

In certain embodiments, the astrocyte enrichment and/or maturation culture medium of various embodiments of the above aspects or any other aspect of the invention delineated herein comprises: i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture; ii) about 5.2-40 ng/mL (e.g., about 10-20 ng/mL) sodium selenite and about 100-300 μM (e.g., about 200 μM) NAC; iii) about 50-200 μg/mL (e.g., about 100 μg/mL) transferrin; iv) about 8-32 μg/mL (e.g., about 16 μg/mL) putrescine; v) about 6-60 ng/mL (e.g., about 30 ng/mL) progesterone; vi) about 0-25 μg/mL insulin; vii) about 2-10 ng/mL (e.g., about 5 ng/mL) HBEGF; viii) about 5-20 ng/mL (e.g., about 10 ng/mL) CNTF; ix) about 10-40 ng/mL (about 20 ng/mL) FGF2; and, x) about 25-100 ng/mL (e.g., about 50 ng/mL) BMP4.

In certain specific embodiments, the astrocyte enrichment medium of the invention comprises: 1:1 DMEM/Neurobasal; 2 mM GLUTAMAX™; 1 mM Sodium Pyruvate; 30 μM NAC; 1×N2 Max; 10 ng/mL BMP4; 10 ng/mL CNTF; 5 ng/mL HBEGF; 20 ng/mL FGF2.

In certain specific embodiments, the astrocyte maturation medium of the invention comprises: 1:1 DMEM/Neurobasal; 2 mM GLUTAMAX™; 1 mM Sodium Pyruvate; 30 μM NAC; 1×N2 Max; 50 ng/mL BMP4; 10 ng/mL CNTF; 5 ng/mL HBEGF; 20 ng/mL FGF2.

In certain embodiments, the astrocyte activation culture medium of the above aspects or any other aspect of the invention delineated herein comprises: i) 1:1 mixture of DMEM/Neurobasal media mixture comprising sodium pyruvate and L-alanyl-L-glutamine dipeptide in the serum-free basal medium; ii) sodium selenite and NAC as said antioxidant; iii) transferrin as said iron carrier; iv) putrescine as said mitogen; v) progesterone as said hormone that activates the progesterone receptor; and, vi) TNFα, IL1a, and/or C1q as said reactive astrocyte drivers.

In certain embodiments, the astrocyte activation culture medium of the above aspects or any other aspect of the invention delineated herein comprises: i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture; ii) about 5.2-40 ng/mL (e.g., about 10-20 ng/mL) sodium selenite and about 20-40 μM (e.g., about 30 μM) NAC; iii) about 50-200 μg/mL (e.g., about 100 μg/mL) transferrin; iv) about 8-32 μg/mL (e.g., about 16 μg/mL) putrescine; v) about 6-60 ng/mL (e.g., about 30 ng/mL) progesterone; vi) about 0-25 μg/mL insulin; and, vii) about 1-5 ng/mL (e.g., about 3 ng/mL) IL-1α, about 15-60 ng/mL (e.g., about 30 ng/mL) TNFα, and about 200-800 ng/mL (e.g., about 400 ng/mL) C1q.

In certain specific embodiments, the reactive astrocyte medium of the invention comprises: 1:1 DMEM/Neurobasal; 2 mM GLUTAMAX™; 1 mM Sodium Pyruvate; 30 μM NAC; 1×N2 Max; 3 ng/mL IL-1α; 30 ng/mL TNFα; 400 ng/mL C1q.

In any of the preceding embodiment, ciliary neurotrophic factor (CNTF) can be substituted with Leukocyte Inhibitory Factor (LIF), Oncostatin M (OSM), or Interleukin-6 (IL6).

In any of the preceding embodiment, Bone Morphogenetic Protein 4 (BMP4) can be substituted with, but not limited by, Bone Morphogenetic Protein 2 (BMP2), Bone Morphogenetic Protein 5 (BMP5), Bone Morphogenetic Protein 6 (BMP6), Bone Morphogenetic Protein 7 (BMP7), Bone Morphogenetic Protein 10 (BMP10), or Bone Morphogenetic Protein 15 (BMP15).

In any of the preceding embodiments, the culture medium of the invention may further comprise an antibiotics, such as PenStrep, a cocktail of both penicillin and streptomycin.

In certain embodiments, the antibiotics comprises neomycin. In certain embodiments, the antibiotics comprises kanamycin. In certain embodiments, the antibiotics comprises antibiotic-antimycotic (Pen, Strep, Amphotericin B, 100×). In certain embodiments, the antibiotics comprises Actinomycin D. In certain embodiments, the antibiotics comprises Ampicillin. In certain embodiments, the antibiotics comprises Carbenicillin. In certain embodiments, the antibiotics comprises Fosmidomycin. In certain embodiments, the antibiotics comprises Gentamycin (10 mg/ml or 50 mg/ml). In certain embodiments, the antibiotics comprises Gentamicin/Amphotericin. In certain embodiments, the antibiotics comprises Penicillin Steptomycin (Pen Strep, such as 5,000 U/ml, or 10,000 U/ml). In certain embodiments, the antibiotics comprises Pen Strep with glutamine (100×). In certain embodiments, the antibiotics comprises Penicillin-Streptomycin-Neomycin (PSN). In certain embodiments, the antibiotics comprises Streptomycin.

In certain embodiments, the antibiotics comprises one or more selection antibiotics, such as Blasticidin, Geneticin (G-418), Hygromycin B, Mycophenolic Acid, Puromycin, Zeocin, Streptomycin Sulfate, Actinomycin D, Ampicillin Sodium Salt, Carbenicillin, Disodium Salt, Kanamycin Sulfate, Neomycin Sulfate, and/or Polymyxin B Sulfate.

### IV. Kits

Another aspect of the invention provides a kit comprising one or more components of any of the culture medium of the invention.

In certain embodiments, the kit is a complete kit, comprising all the components necessary to prepare a culture medium of the invention, wherein at least one of the components is not mixed with the others. In certain embodiments, the culture medium of the invention is a resting astrocyte medium of the invention. In certain embodiments, the culture medium of the invention is an astrocyte enrichment medium of the invention. In certain embodiments, the culture medium of the invention is an astrocyte maturation medium of the invention. In certain embodiments, the culture medium of the invention is an astrocyte activation medium of the invention.

In certain embodiments, the kit is a partial kit, comprising all the components necessary to prepare a culture medium of the invention, except for one or more commonly available commercial products such as the serum-free basal medium (e.g., DMEM, Neurobasal medium etc.), L-glutamine source (such as GLUTAMAX™), N2 Max, etc. In certain embodiments, the culture medium of the invention is a resting astrocyte medium of the invention. In certain embodiments, the culture medium of the invention is an astrocyte enrichment medium of the invention. In certain embodiments, the culture medium of the invention is an astrocyte maturation medium of the invention. In certain embodiments, the culture medium of the invention is an astrocyte activation medium of the invention.

In certain embodiments, the kit of the invention further comprises an instruction for making the culture medium of the invention, including steps to take to mix different components of the medium under selected/optimal conditions, timing of mixing different components, and/or temporary storage conditions for pre-mixed components.

The instruction can be physical printed material, or can be a link to an internet website (scan barcode, web URL, etc.).

### V. Cultured Cells/Astrocytes

Another aspect of the invention provides a cell culture, comprising any one of the medium of the invention, and an astrocyte-containing cell population.

In certain embodiments, the medium of the invention is a resting astrocyte culture medium of the invention.

In certain embodiments, the medium of the invention is an astrocyte enrichment culture medium of the invention.

In certain embodiments, the medium of the invention is an astrocyte maturation culture medium of the invention.

In certain embodiments, the medium of the invention is an astrocyte activation culture medium of the invention.

In certain embodiments, the astrocyte-containing cell population is a population of cells obtained from a neuronal tissue, such as a CNS tissue. In certain embodiments, the (CNS) tissue is cortex, corpus callosum, hippocampus, midbrain, pons, medulla, brainstem, cerebellum, and/or spinal cord.

In certain embodiments, the CNS tissue is from a patient having, suspect of having, or at high risk of having Multiple Sclerosis (MS), Alzheimer's Disease (AD), Huntington's diseases (HD), Amyotrophic Lateral Sclerosis (ALS), and Parkinson's Disease (PD).

In certain embodiments, the cell culture is a model system (e.g., an in vitro model system) for a neurological disease, such as Multiple Sclerosis (MS), Alzheimer's Disease (AD), Huntington's diseases (HD), Amyotrophic Lateral Sclerosis (ALS), and Parkinson's Disease (PD).

In certain embodiments, the CNS tissue is from a mammal such as a human, or a rodent, such as mouse (such as C57BL/6, BALB/c), rat, hamster, Guinea pig, or gerbil.

In certain embodiments, the astrocyte-containing cell population is a mixture of cells derived from differentiation of stem cells or progenitor cells. In certain embodiments, the stem cells or progenitor cells include oligodendrocyte progenitors (OPCs), glial progenitors (GPCs), glial restricted progenitors (GRPs), and induced pluripotent stem cells (iPSCs), iPS cells, embryonic stem cells (ESCs), and epiblast stem cells (EpiSCs).

In certain embodiments, the astrocyte-containing cell population is a mixture of cells resulting from reprogramming of somatic cells.

In certain embodiments, the medium of the invention is a resting astrocyte culture medium of the invention, and the astrocyte-containing cell population is substantially pure astrocytes (e.g., having no more than 20%, 15%, 10%, 5%, 4%, 3%, 2%, 1% non-astrocytes, such as microglia cells, neurons, or dendritic cells.

In certain embodiments, the substantially pure astrocytes in the culture substantially lack expression of oligodendrocyte markers, such as Sox10 or Mbp.

In certain embodiments, the substantially pure astrocytes in the culture substantially lack expression of neuronal markers, such as Snap25 or Nef1.

In certain embodiments, the substantially pure astrocytes in the culture substantially lack expression of microglia markers, such as Cd68 or Tmem119.

In certain embodiments, the substantially pure astrocytes in the culture has been in cryopreservation.

In certain embodiments, the substantially pure astrocytes in the culture is thawed from cryopreservation.

In a related aspect, the invention provides a cryopreserved culture of enriched astrocytes, obtained from a population of astrocyte-containing cells by: (a) culturing single cells of the population of astrocyte-containing cells, in said astrocyte enrichment culture medium of the invention, with fresh medium change about every 2 days until a confluent culture is formed, in order to enrich for proliferating/proliferated astrocytes in the single cell culture; and, (b) cryopreserving the confluent culture.

A related aspect of the invention provides a cryopreserved substantially pure culture of resting astrocytes, wherein the substantially pure culture of resting astrocytes are obtained by the method of the invention.

In certain embodiments, the population of astrocyte-containing cells are derived from mammalian neuronal tissues, and differentiated or differentiating stem cells or progenitor cells.

In certain embodiments, the mammalian neuronal tissues are isolated from a central nervous system (CNS) tissue, such as cortex, corpus callosum, hippocampus, midbrain, pons, medulla, brainstem, cerebellum, and/or spinal cord.

In certain embodiments, the differentiated or differentiating stem cells or progenitor cells comprise oligodendrocyte progenitors (OPCs), glial progenitors (GPCs), glial restricted progenitors (GRPs), and/or neural progenitors (NPCs).

In certain embodiments, the stem cells or progenitor cells comprise induced pluripotent stem cells (iPSCs), embryonic stem cells (ESCs), neural stem cells (NSCs), iPS cells, and/or epiblast stem cells (EpiSCs).

In certain embodiments, the mammalian neuronal tissues and/or cells are human.

In certain embodiments, the mammalian neuronal tissues and/or cells are non-human.

In certain embodiments, the non-human mammalian neuronal tissues and/or cells are from, but not limited to, mouse or rat.

### VI. Method of Isolating/Purifying Astrocytes

Another aspect of the invention provides a method of isolating a substantially pure culture of resting astrocytes from a population of astrocyte-containing cells, the method comprising: (1) culturing single cells of the population of astrocyte-containing cells, in said astrocyte enrichment culture medium of various embodiments of the above aspects or any other aspect of the invention delineated herein, with fresh medium change about every 2 days until a confluent culture is formed, in order to enrich for proliferating/proliferated astrocytes in the single cell culture; (2) passaging the confluent culture once in said astrocyte maturation culture medium of various embodiments of the above aspects or any other aspect of the invention delineated herein for about 2 days to permit astrocyte maturation, or optionally cryopreserving the confluent culture before said passaging; and, (3) replacing the astrocyte maturation culture medium after about 2 days, with said resting astrocyte culture medium of various embodiments of the above aspects or any other aspect of the invention delineated herein for about 3 more days, thereby producing the substantially pure culture of resting astrocytes.

In certain embodiments, the population of astrocyte-containing cells are derived from mammalian neuronal tissues, and differentiated or differentiating stem cells or progenitor cells.

For example, in certain embodiments, the population of astrocyte-containing cells are derived from mammalian neuronal tissues, which can be isolated from a central nervous system (CNS) tissue, such as cortex, corpus callosum, hippocampus, midbrain, pons, medulla, brainstem, cerebellum, and/or spinal cord.

In certain embodiments, the mammalian neuronal tissues are human neuronal tissues, such as human CNS (e.g., brain and spinal cord) or parts thereof, such as cortex, corpus callosum, hippocampus, midbrain, pons, medulla, brainstem, cerebellum, and/or spinal cord. The human may have had a neurodegenerative disease, such as Multiple Sclerosis (MS), Alzheimer's Disease (AD), Huntington's diseases (HD), Amyotrophic Lateral Sclerosis (ALS), and Parkinson's Disease (PD).

In certain embodiments, the human CNS tissue is obtained post mortem.

In certain embodiments, the mammalian neuronal tissues are rodent neuronal tissues, such as rodent CNS (e.g., brain and spinal cord) or parts thereof, such as cortex, corpus callosum, hippocampus, midbrain, pons, medulla, brainstem, cerebellum, and/or spinal cord. The rodent can be mouse (such as C57BL/6, BALB/c), rat, hamster, Guinea pig, or gerbil. The mouse may be an animal model of a neurodegenerative disease, such as Multiple Sclerosis (MS), Alzheimer's Disease (AD), Huntington's diseases (HD), Amyotrophic Lateral Sclerosis (ALS), and Parkinson's Disease (PD).

In certain embodiments, the rodent (e.g., mouse) neuronal tissue is obtained from young postnatal animal, such as postnatal day 1, 2, 3, or 4.

In certain embodiments, the astrocytes isolated/purified by the methods and media of the invention are primary astrocytes.

In certain embodiments, the mammalian neuronal tissues are enriched for gray matter, such as frontal cortex.

In certain embodiments, the mammalian neuronal tissues are enriched for white matter, such as corpus callosum.

In certain embodiments, the population of astrocyte-containing cells are a mixture of cells derived from differentiation of stem cells or progenitor cells. In certain embodiments, the stem cells or progenitor cells include oligodendrocyte progenitors (OPCs), glial progenitors (GPCs), glial restricted progenitors (GRPs), and induced pluripotent stem cells (iPSCs), embryonic stem cells (ESCs), and epiblast stem cells (EpiSCs).

In certain embodiments, the population of astrocyte-containing cells are a mixture of cells resulting from reprogramming of somatic cells.

In certain embodiments, according to the method of the invention, the population of astrocyte-containing cells are first dissociated into single cells. The dissociation step may be physical, chemical, or enzymatic, or a combination thereof.

In certain embodiments, the dissociation step can be performed using commercial kits, such as the Miltenyi Tumor Dissociation Kit (Cat. No. 130-095-929, Miltenyi Biotec). The Tumor Dissociation Kit facilitates gentle and rapid generation of single-cell suspensions from primary human tissues, such as tumor tissues. The kit is ideally suited for the time-saving and reproducible preparation of single-cell suspensions in combination with the gentleMACS™ Dissociators. Though the kit is optimized for high yield of tumor cells and tumor-infiltrating lymphocytes, it has been tested to be suitable for the method of the invention.

In certain embodiments, the dissociation step is carried out with the Miltenyi Neural Tissue Dissociation Kit (P) (Cat #: 130-092-628); the Miltenyi Neural Tissue Dissociation Kit (T) (Cat #: 130-093-231); or the Homemade Papain Based Dissociation (Ben Emery and Jason C. Dugas. Purification of Oligodendrocyte Lineage Cells from Mouse Coritices by Immunopanning. Cold Spring Harbor Protocols 2013) [lx Earle Balanced Salt Solution; 0.12 M NaCl; 5.4 mM KCl; 1 mM NaH₂PO₄; 0.1% Glucose; 0.0005% Phenol Red; 1 mM MgSO₄; 0.46% Glucose; 2 mM EGTA; 26 mM NaHCO₃; 200 units of papain enzyme (this could be substituted with trypsin, dispase, or collagenase); 2500 units of DNase I].

In certain embodiments, the dissociation comprises dissociating the mammalian neuronal tissues using enzymatic digestion with mechanical trituration.

In certain embodiments, enzymatic digestion is performed with papain, trypsin, dispase, and/or collagenase.

In certain embodiments, resting astrocytes: (1) are positive for any one or more of the astrocyte markers selected from GFAP, AQP4, GLT-1, VIMENTIN, GLAST, and ALDH1L1; (2) do not express Lcn2, Steap4, and/or Cxc110; (3) exhibit a stellate morphology; and/or (4) only express mature astrocyte genes (such as Gja1 or Sox9), with no expression of oligodendrocyte genes (such as Sox10 and Mbp), microglia genes (such as Cd68 and Tmem119), and neuron markers (such as Nef1 and Snap25).

In certain embodiments, said resting astrocytes take up exogenous glutamate. In certain embodiments, said resting astrocyte take up at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of exogenous glutamate.

In some embodiments, said resting astrocyte take up between about 10% to about 90%, between about 15% to about 90%, between about 20% to about 90%, between about 25% to about 90%, between about 30% to about 90%, between about 35% to about 90%, between about 40% to about 90%, between about 45% to about 90%, between about 50% to about 90%, between about 55% to about 90%, between about 60% to about 90%, between about 65% to about 90%, between about 70% to about 90%, between about 75% to about 90%, between about 80% to about 90%, between about 85% to about 90%, between about 10% to about 85%, between about 15% to about 85%, between about 20% to about 85%, between about 25% to about 85%, between about 30% to about 85%, between about 35% to about 85%, between about 40% to about 85%, between about 45% to about 85%, between about 50% to about 85%, between about 55% to about 85%, between about 60% to about 85%, between about 65% to about 85%, between about 70% to about 85%, between about 75% to about 85%, between about 80% to about 85%, between about 10% to about 80%, between about 15% to about 80%, between about 20% to about 80%, between about 25% to about 80%, between about 30% to about 80%, between about 35% to about 80%, between about 40% to about 80%, between about 45% to about 80%, between about 50% to about 80%, between about 55% to about 80%, between about 60% to about 80%, between about 65% to about 80%, between about 70% to about 80%, between about 75% to about 80%, between about 10% to about 75%, between about 15% to about 75%, between about 20% to about 75%, between about 25% to about 75%, between about 30% to about 75%, between about 35% to about 75%, between about 40% to about 75%, between about 45% to about 75%, between about 50% to about 75%, between about 55% to about 75%, between about 60% to about 75%, between about 65% to about 75%, between about 70% to about 75%, between about 10% to about 70%, between about 15% to about 70%, between about 20% to about 70%, between about 25% to about 70%, between about 30% to about 70%, between about 35% to about 70%, between about 40% to about 70%, between about 45% to about 70%, between about 50% to about 70%, between about 55% to about 70%, between about 60% to about 70%, between about 65% to about 70%, between about 10% to about 65%, between about 15% to about 65%, between about 20% to about 65%, between about 25% to about 65%, between about 30% to about 65%, between about 35% to about 65%, between about 40% to about 65%, between about 45% to about 65%, between about 50% to about 65%, between about 55% to about 65%, between about 60% to about 65%, between about 10% to about 60%, between about 15% to about 60%, between about 20% to about 60%, between about 25% to about 60%, between about 30% to about 60%, between about 35% to about 60%, between about 40% to about 60%, between about 45% to about 60%, between about 50% to about 60%, between about 55% to about 60%, between about 10% to about 55%, between about 15% to about 55%, between about 20% to about 55%, between about 25% to about 55%, between about 30% to about 55%, between about 35% to about 55%, between about 40% to about 55%, between about 45% to about 55%, between about 50% to about 55%, between about 10% to about 50%, between about 15% to about 50%, between about 20% to about 50%, between about 25% to about 50%, between about 30% to about 50%, between about 35% to about 50%, between about 40% to about 50%, between about 45% to about 50%, between about 10% to about 45%, between about 15% to about 45%, between about 20% to about 45%, between about 25% to about 45%, between about 30% to about 45%, between about 35% to about 45%, between about 40% to about 45%, between about 10% to about 40%, between about 15% to about 40%, between about 20% to about 40%, between about 25% to about 40%, between about 30% to about 40%, between about 35% to about 40%, between about 10% to about 35%, between about 15% to about 35%, between about 20% to about 35%, between about 25% to about 35%, between about 30% to about 35%, between about 10% to about 30%, between about 15% to about 30%, between about 20% to about 30%, between about 25% to about 30%, between about 10% to about 25%, between about 15% to about 25%, between about 20% to about 25%, between about 10% to about 20%, or between about 15% to about 20% of exogenous glutamate.

In certain embodiments, said resting astrocytes have increased phagocytic activity when compared to reactive astrocytes. In certain embodiments, said resting astrocytes have at least 1.2 fold, at least 1.3 fold, at least 1.4 fold, at least 1.5 fold, at least 1.6 fold, at least 1.7 fold, at least 1.8 fold, at least 1.9 fold, at least 2.0 fold increased phagocytic activity when compared to reactive astrocytes.

In certain embodiments, said resting astrocytes have between about 1.1 fold to about 2.5 fold, between about 1.2 to about 2.5 fold, between about 1.3 fold to about 2.5 fold, between about 1.4 fold to about 2.5 fold, between about 1.5 fold to about 2.5 fold, between about 1.6 fold to about 2.5 fold, between about 1.7 fold to about 2.5 fold, between about 1.8 fold to about 2.5 fold, between about 1.9 fold to about 2.5 fold, between about 2.0 fold to about 2.5 fold, between 2.1 fold to about 2.5 fold, between 2.2 fold to about 2.5 fold, between 2.3 fold to about 2.5 fold, between about 2.4 to about 2.5 fold, between about 1.1 fold to about 2.4 fold, between about 1.2 to about 2.4 fold, between about 1.3 fold to about 2.4 fold, between about 1.4 fold to about 2.4 fold, between about 1.5 fold to about 2.4 fold, between about 1.6 fold to about 2.4 fold, between about 1.7 fold to about 2.4 fold, between about 1.8 fold to about 2.4 fold, between about 1.9 fold to about 2.4 fold, between about 2.0 fold to about 2.4 fold, between 2.1 fold to about 2.4 fold, between 2.2 fold to about 2.4 fold, between 2.3 fold to about 2.4 fold, between about 1.1 fold to about 2.3 fold, between about 1.2 to about 2.3 fold, between about 1.3 fold to about 2.3 fold, between about 1.4 fold to about 2.3 fold, between about 1.5 fold to about 2.3 fold, between about 1.6 fold to about 2.3 fold, between about 1.7 fold to about 2.3 fold, between about 1.8 fold to about 2.3 fold, between about 1.9 fold to about 2.3 fold, between about 2.0 fold to about 2.3 fold, between 2.1 fold to about 2.3 fold, between 2.2 fold to about 2.3 fold, between about 1.1 fold to about 2.1 fold, between about 1.2 to about 2.1 fold, between about 1.3 fold to about 2.1 fold, between about 1.4 fold to about 2.1 fold, between about 1.5 fold to about 2.1 fold, between about 1.6 fold to about 2.1 fold, between about 1.7 fold to about 2.1 fold, between about 1.8 fold to about 2.1 fold, between about 1.9 fold to about 2.1 fold, between about 2.0 fold to about 2.1 fold, between about 1.1 fold to about 2.0 fold, between about 1.2 to about 2.0 fold, between about 1.3 fold to about 2.0 fold, between about 1.4 fold to about 2.0 fold, between about 1.5 fold to about 2.0 fold, between about 1.6 fold to about 2.0 fold, between about 1.7 fold to about 2.0 fold, between about 1.8 fold to about 2.0 fold, between about 1.9 fold to about 2.0 fold, between about 1.1 fold to about 1.9 fold, between about 1.2 to about 1.9 fold, between about 1.3 fold to about 1.9 fold, between about 1.4 fold to about 1.9 fold, between about 1.5 fold to about 1.9 fold, between about 1.6 fold to about 1.9 fold, between about 1.7 fold to about 1.9 fold, between about 1.8 fold to about 1.9 fold, between about 1.1 fold to about 1.8 fold, between about 1.2 to about 1.8 fold, between about 1.3 fold to about 1.8 fold, between about 1.4 fold to about 1.8 fold, between about 1.5 fold to about 1.8 fold, between about 1.6 fold to about 1.8 fold, between about 1.7 fold to about 1.8 fold, between about 1.1 fold to about 1.7 fold, between about 1.2 to about 1.7 fold, between about 1.3 fold to about 1.7 fold, between about 1.4 fold to about 1.7 fold, between about 1.5 fold to about 1.7 fold, between about 1.6 fold to about 1.7 fold, between about 1.1 fold to about 1.6 fold, between about 1.2 to about 1.6 fold, between about 1.3 fold to about 1.6 fold, between about 1.4 fold to about 1.6 fold, between about 1.5 fold to about 1.6 fold, between about 1.1 fold to about 1.5 fold, between about 1.2 to about 1.5 fold, between about 1.3 fold to about 1.5 fold, between about 1.4 fold to about 1.5 fold, between about 1.1 fold to about 1.4 fold, between about 1.2 to about 1.4 fold, between about 1.3 fold to about 1.4 fold, between about 1.1 fold to about 1.3 fold, between about 1.2 to about 1.3 fold, or between about 1.1 fold to about 1.2 fold increased phagocytic activity when compared to reactive astrocytes.

In certain embodiments, said resting astrocytes express one or more reactive astrocyte markers when infected with Thieler's murine encephalomyelitis virus (TMEV), thereby becoming reactive astrocytes. In certain embodiments, the reactive astrocyte marker is guanylate binding protein 2 (GBP2). In certain embodiments, said resting astrocytes infected with TMEV express at least 70%, at least 75%, at least 80%, at least 85%, at least 90%, at least 95%, or at least 98% more reactive astrocyte marker compared to uninfected resting astrocytes.

In certain embodiments, said resting astrocytes infected with TMEV express between about 70% to about 100%, between about 75% to about 100%, between about 80% to about 100%, between about 85% to about 100%, between about 90% to about 100%, between about 95% to about 100%, between about 70% to about 95%, between about 75% to about 95%, between about 80% to about 95%, between about 85% to about 95%, between about 90% to about 95%, between about 70% to about 90%, between about 75% to about 90%, between about 80% to about 90%, between about 85% to about 90%, between about 70% to about 85%, between about 75% to about 85%, between about 80% to about 85%, between about 70% to about 80%, between about 75% to about 80%, or between about 70% to about 75% more reactive astrocyte marker compared to uninfected resting astrocytes.

In certain embodiments, the method produces substantially pure resting astrocytes. In certain embodiments, at least about 90%, 95%, 96%, 97%, 98%, 99% or more of the cells in the substantially pure culture are resting astrocytes.

In certain embodiments, the substantially pure culture of resting astrocytes comprises about 10⁶, 10⁷, 10⁸, 10⁹, 10¹⁰, 10¹¹, 10¹² or 10¹³ resting astrocytes.

In certain embodiments, prior to culturing the single cells in said astrocyte enrichment culture medium, the method further comprises culturing the single cells for about 24 hours in a media consisting of Dulbecco's Modified Eagle Medium/Nutrient Mixture F-12 (DMEM/F-12), N-2 Supplement, B-27 Supplement, GLUTAMAX™ Supplement, Penicillin-Streptomycin, and FGF-2.

### VII. Method of Screening

Another aspect of the invention provides a method of identifying a compound that inhibits reactive astrocyte formation from resting astrocytes, the method comprising: contacting a substantially pure culture of resting astrocytes obtained by the method of various embodiments of the above aspects or any other aspect of the invention delineated herein, or a thawed culture of the cryopreserved substantially pure culture of resting astrocytes of various embodiments of the above aspects or any other aspect of the invention delineated herein, with a candidate compound from a library of compounds, in the presence of the astrocyte activation culture medium of various embodiments of the above aspects or any other aspect of the invention delineated herein, for a sufficient period of time (e.g., 24 hours), before determining the expression level of a marker gene for reactive astrocyte; wherein the candidate compound that statistically significantly decreased the number of marker gene-positive reactive astrocytes by greater than 50%, 60%, 70%, 80%, 90%, 95% or more compared to vehicle (e.g., a solvent for the candidate compound such as DMSO) control is identified as the compound that inhibits reactive astrocyte formation.

In certain embodiments, the marker gene comprises one or more of GBP2 PSMB8, C3, H2-D1, H2-T23, SERPING1, and IIGP1.

In certain embodiments, the method further comprising determining the expression level of the marker gene.

In certain embodiments, the candidate compound is not significantly toxic to astrocytes (e.g., the candidate compound does not decrease the counted number of live cells by greater than 30% compared to the vehicle control).

In certain embodiments, the method has a z-prime score of 0.6, 0.7 or higher.

In certain embodiments, the resting astrocytes are contacted in 96-well, 384-well, or 1,536-well tissue culture plates in a high throughput platform suitable for multiplex screening.

In certain embodiments, the resting astrocytes are contacted by the candidate compound before prior to (e.g., 1 hour prior to), simultaneously with, or subsequent to (e.g., within 30 min of) contacting with the astrocyte activation culture medium.

In certain embodiments, the astrocyte activation culture medium comprises about 1-5 ng/mL (e.g., about 3 ng/mL) IL-1α, about 15-60 ng/mL (e.g., about 30 ng/mL) TNFα, and about 200-800 ng/mL (e.g., about 400 ng/mL) C1q.

In certain embodiments, the method further comprising confirming that the candidate compound does not substantially affect (e.g., decrease) the expression level of a pan-astrocyte marker (such as vimentin).

In certain embodiments, the library of compounds comprise histone deacetylase (HDAC) inhibitors, proteasome inhibitors, and inhibitors of NFκB signaling.

With the invention generally described above, the following non-limiting examples are for illustrative purpose, and should not be construed as limiting in any respect.

### EXAMPLES

### Example 1 Isolation/Purification of Resting Astrocytes

This example demonstrates the isolation/purification of astrocytes using the culture media of the invention from primary tissues, such as neuronal tissues (e.g., brain). Similar methods can be used to isolate astrocytes from other sources, such as differentiated stem cells or other cell mixture.

Brains from mice of the C57BL/6 strain were extracted at postnatal day 2 (P2). A gross dissection of these brains was performed to isolate the cortices, which includes astrocytes. These cortices were dissociated following the protocols found in the Miltenyi Tumor Dissociation Kit (130-095-929, Miltenyi). After dissociation, cells were plated in flat-bottomed plastic flasks coated with a substrate of poly-L-ornithine (Sigma, P3655) and laminin (Sigma, L2020). Cells were cultured for 24 hours in media consisting of Dulbecco's Modified Eagle Medium/Nutrient Mixture F-12 (DMEM/F-12; 11320033, ThermoFisher Scientific), N-2 Supplement (17502048, ThermoFisher Scientific), B-27 Supplement (17504044, ThermoFisher Scientific), GLUTAMAX™ Supplement (35050079, ThermoFisher Scientific), Penicillin-Streptomycin (15070063, ThermoFisher Scientific), and FGF-2.

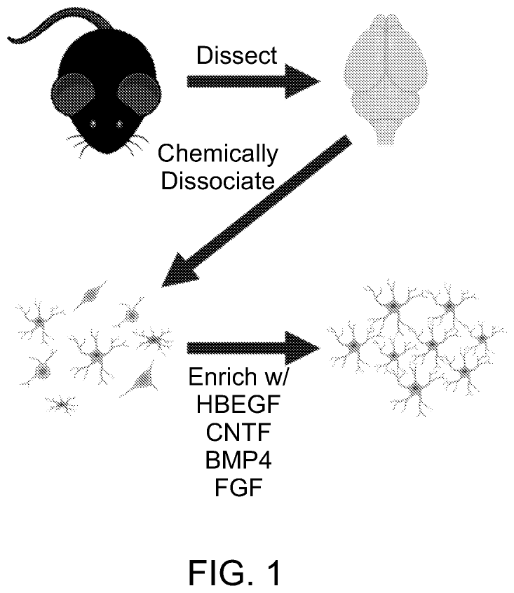

After 24 hours, the cells were switched to astrocyte enrichment media comprised of DMEM (11960044, ThermoFisher Scientific), Neurobasal Medium (21103049, ThermoFisher Scientific), GLUTAMAX™ Supplement, Sodium Pyruvate (11360070, ThermoFisher Scientific), N-2max Supplement (R&D #AR009), N-acetyl cysteine (Sigma #A8199), Penicillin-Streptomycin (ThermoFisher Scientific #15070-063), 5 ng/mL HB-EGF (R&D Systems #259-HE-050), 10 ng/mL CNTF (R&D Systems #557-NT-010), 10 ng/mL BMP4 (R&D Systems #314-BP-050), and 20 ng/mL FGF2 (R&D Systems #233-FB-01M) to proliferate. Media was changed every 48 hours. Once confluent astrocytes were either cryopreserved or passaged once and then cryopreserved. See a schematic drawing in FIG. 1 that illustrates the general process.

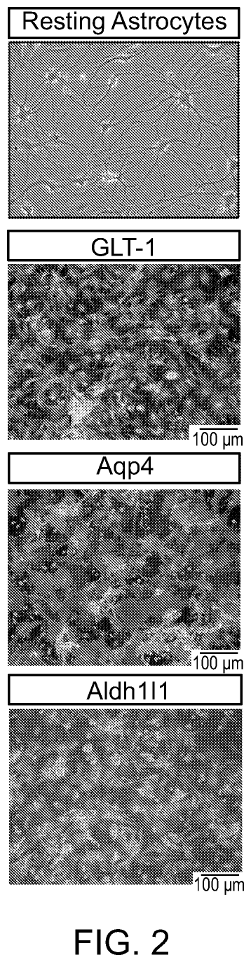

As shown in FIG. 2, phase contrast and immunofluorescence images of resting astrocytes, showing resting astrocytes exhibiting a stellate morphology similar to their in vivo morphology. Resting astrocytes also express canonical mature astrocyte markers GFAP, AQP4, GLT-1, and ALDH1L1.

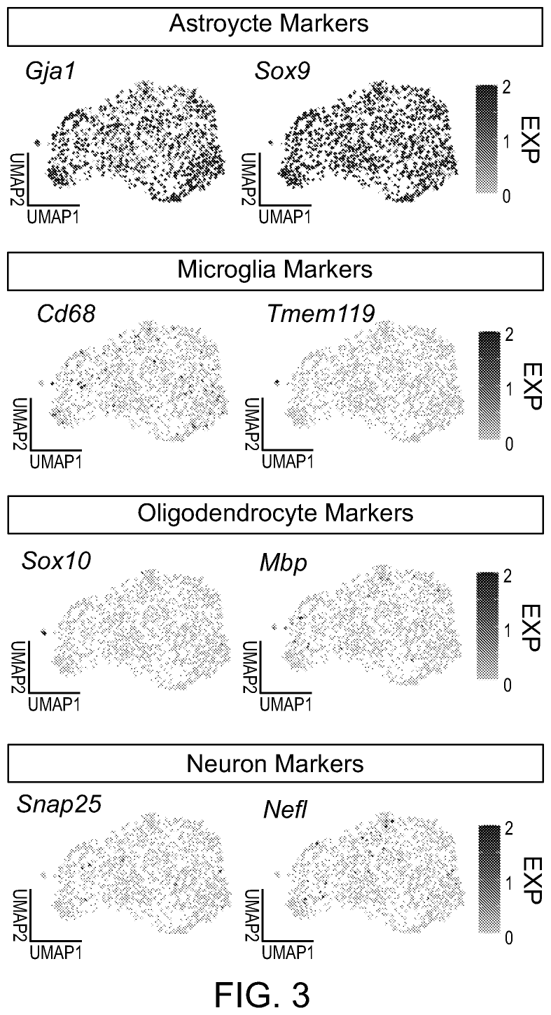

FIG. 3 shows that the resting astrocyte cultures of the invention are highly pure, as demonstrated by single-cell RNAseq data. Single-cell RNAseq of resting astrocyte cultures show that only mature astrocyte genes are expressed with no expression of oligodendrocyte, microglia, or neuron markers.

Single-cell RNAseq was performed by lifting resting and reactive astrocytes from culture plates using TrypLE (ThermoFisher, 12563011). Cells were collected in 1×PBS+1% bovine serum albumin (BSA). Cells were then spun down at 1000 rpm for 10 minutes. After which cells were washed once with 1×PBS+1% BSA before being filtered through 40 μm FLowMi Tip Strainers (VWR, 10032-802). Cells were then diluted with 1×PBS+1% BSA and loaded onto the Chromium 10× Controller according to manufacturer's instructions. Following partitioning of single-cells in gel bead emulsions, reverse-transcriptions and library preparation were performed according to 10× Single Cell 3′ v2 chemistry kit instructions (v2 kit since discontinued). Finally, libraries were sequenced on an Illumina HiSeq2500 with paired-end 50 bp reads, and a target sequence depth of 50,000 reads per cell. Sequence data was first processed by 10× Cell Ranger v2.0 using default settings to generate a gene expression matrix and then all downstream analysis was performed with the R package Seurat v4.012.

To conduct a terminal experiment, cryopreserved astrocytes were removed from liquid nitrogen storage and thawed in astrocyte maturation media (DMEM, Neurobasal Medium, GLUTAMAX™ Supplement, Sodium Pyruvate, N-2 Supplement, N-acetyl cysteine) supplemented with 5 ng/mL HB-EGF, 10 ng/mL CNTF, 50 ng/mL BMP4, and 20 ng/mL FGF2 for 48 hours followed by resting astrocyte media containing only 5 ng/mL HB-EGF for another 72 hours. After this five-day thawing and maturation protocol, experimental treatments could be applied to these mature astrocytes in culture.

Miltenyi Tumor Dissociation Kit (Cat #: 130-095-929) can be substituted with: Miltenyi Neural Tissue Dissociation Kit (P) (Cat #: 130-092-628); Miltenyi Neural Tissue Dissociation Kit (T) (Cat #: 130-093-231); or Homemade Papain Based Dissociation (Ben Emery and Jason C. Dugas. Purification of Oligodendrocyte Lineage Cells from Mouse Coritices by Immunopanning. Cold Spring Harbor Protocols 2013) [1× Earle Balanced Salt Solution; 0.12 M NaCl; 5.4 mM KCl; 1 mM NaH₂PO₄; 0.1% Glucose; 0.0005% Phenol Red; 1 mM MgSO₄; 0.46% Glucose; 2 mM EGTA; 26 mM NaHCO₃; 200 units of papain enzyme (this could be substituted with trypsin, dispase, or collagenase); 2500 units of DNase I].

### Example 2 Human Astrocyte Generation

Human induced pluripotent stem cells (iPSCs) were cultured to differentiate into astrocytes as described by Perriot et al. (2018). In short, iPSC colonies were placed in neural induction media for 10 days until neural rosettes could be picked, dissociated, and plated in a glial expansion medium. These cells were allowed to proliferate and become a homogenous population of glial progenitor cells (GPCs) over eight passages on poly-L-ornithine and laminin-coated plates.

These GPCs were then passaged onto a Matrigel-coated plate to culture in an astrocyte induction media for two weeks, which was followed by culturing for another four weeks in an astrocyte maturation medium.

### Example 3 Induction of Reactive Astrocytes

Damaging reactive astrocytes are present in many neurological diseases. This example demonstrates that such damaging reactive state can be mimicked in vitro through the exogenous delivery of proinflammatory cytokines Tumor Necrosis Factor-alpha (TNFα), interleukin 1-alpha (IL1α), and complement component 1q (C1q). Exposure of astrocytes to these factors results in the formation of damaging reactive astrocytes that exhibit a cell state specific gene expression signature.

### Example 4 Screening for Inhibitors of Reactive Astrocytes

To identify chemical compounds capable of blocking the formation of damaging reactive astrocytes, resting astrocytes were thawed and plated onto 384-well plates as described above at a density of 500 cells/mm². A Perkin Elmer Janus G3 Varispan Automated Workstation was then used to treat cells with small-molecules with one molecule per well at a concentration of 2 μM in 384-well plates, followed one hour later by the addition of reactive astrocyte media containing 3 ng/mL IL-1α (Sigma #I3901), 400 ng/mL C1q (MyBioSource #MBS143105), and 30 ng/mL TNFα (R&D Systems #210-TA-020) (Table 1).

After incubation for 24 hours, the cells were fixed using 4% paraformaldehyde and stained for GBP2 (Proteintech #11854-1-AP) using the procedure detailed in the immunocytochemistry section below. Then imaged using the PerkinElmer Operetta CLS High-Content Analysis System. Images were analyzed using automated PerkinElmer Columbus Image Analysis Software. For analysis toxic chemicals were first removed, a chemical was considered toxic if it decreased the counted number of live cells in the well by greater than 30% compared to reactive astrocyte plus DMSO vehicle control wells. Hits were then determined as those compounds that decreased the number of GBP2 positive reactive astrocytes by greater than 80% compared to reactive astrocytes plus DMSO vehicle control wells.

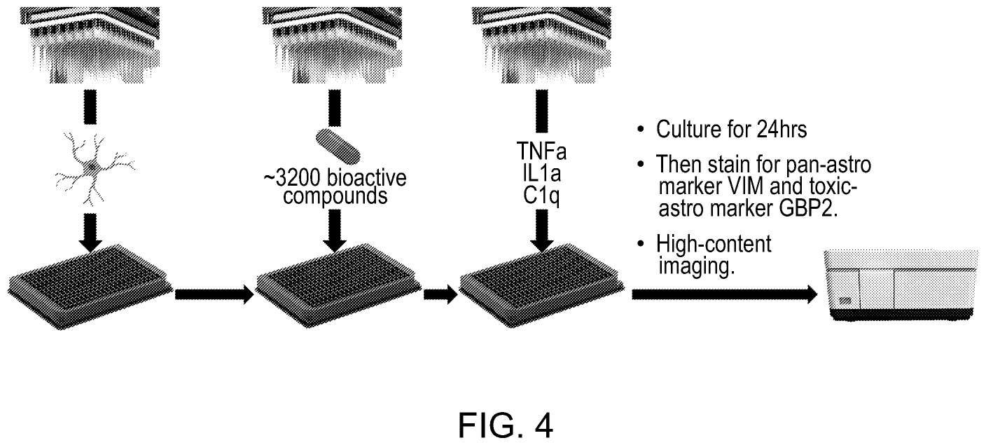

This primary screen had high reproducibility across independent compound plates and a z-prime score of 0.734, which is excellent for a cell-based screen. This robust primary screen resulted in the discovery of 93 potential inhibitors of damaging reactive astrocyte polarization. These 93 primary hits were filtered using a secondary dose curve screen. The result of this dose curve screen is the validation of 33 compounds across 14 compound target classes that all inhibit the formation of damaging reactive astrocytes polarized by TNFα, IL1α, and C1q. None of these compounds have been previously described for this function. See FIG. 4.

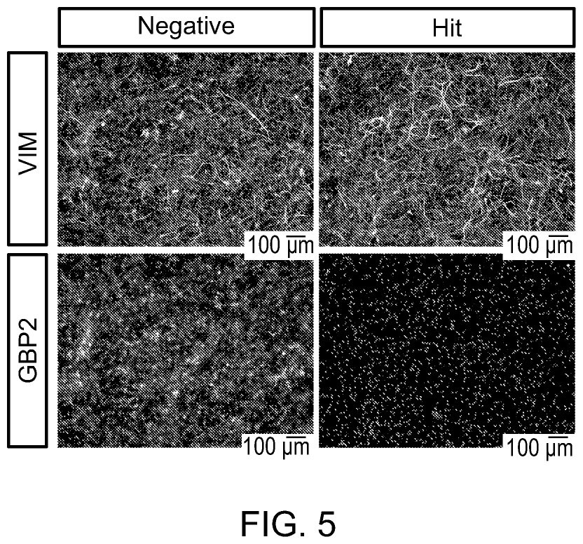

Results of an exemplary ineffective negative compound, and an effective hit compound were shown in FIG. 5. Specifically, the non-toxic ineffective negative compound does not decrease the expression of guanylate binding protein 2 (GBP2) in astrocytes exposed to reactive astrocyte media. The effective hit compound decreased the expression of GBP2 in astrocytes exposed to reactive astrocyte media, was not toxic, and did not decrease the total number of cells in the well by greater than 30% compared to control wells, and did not affect the expression of the counterstain for vimentin (VIM).

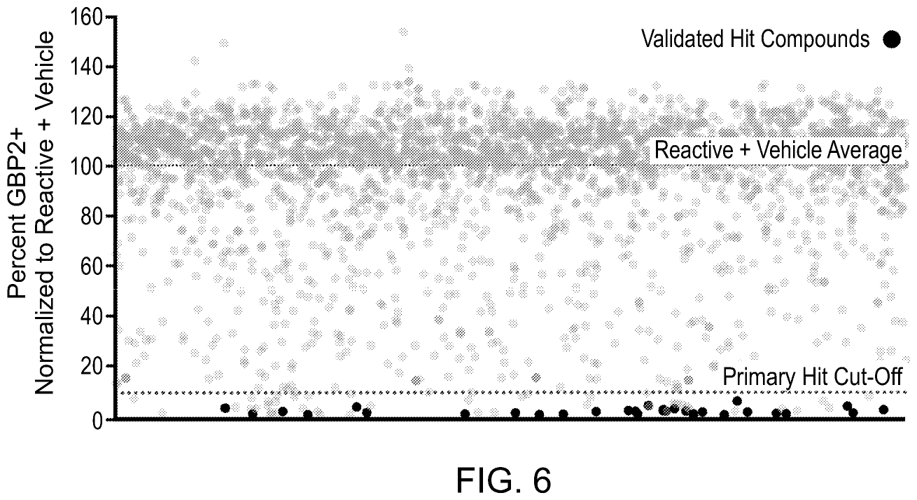

The primary screen results were shows in FIG. 6 as a dot plot. Each gray dot represents a non-toxic compound that was not validated as an inhibitor of reactive astrocyte formation. Hit compounds in black were those that decreased the formation of GBP2+ reactive astrocytes by 80% or more, and were further validated in secondary dose curve assays.

### Example 4 Activities of Generated Resting Astrocytes

Resting astrocytes generated with the methods described above took up glutamate. This shows that the astrocytes generated with the methods described herein recapitulate a canonical function of astrocytes in vivo.

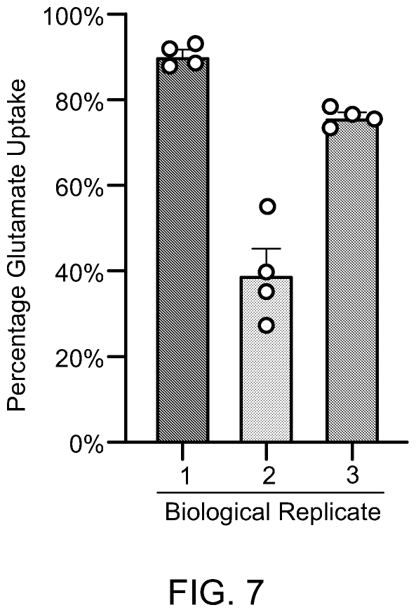

Specifically, resting astrocytes cultured in glutamate-free media were exposed to a known concentration of exogenous glutamate, incubated for 6 hrs, and then the amount of glutamate uptake by the resting astrocytes was measured. Three biological replicates were tested. Glutamate uptake by generated resting astrocytes was between about 40% to about 85% of the known concentration of exogenous glutamate (FIG. 7).

Resting astrocytes generated with the methods described herein phagocytosed myelin debris and this function is decreased in reactive astrocytes. This data shows that resting astrocytes generated with the methods described herein recapitulate a canonical function of astrocytes in vivo. Meanwhile, and similar to what has been reported by others, reactive astrocytes generated with the methods as described herein have decreased phagocytic capabilities. Compared to resting astrocytes, reactive astrocytes show decreased phagocytosis of myelin debris.

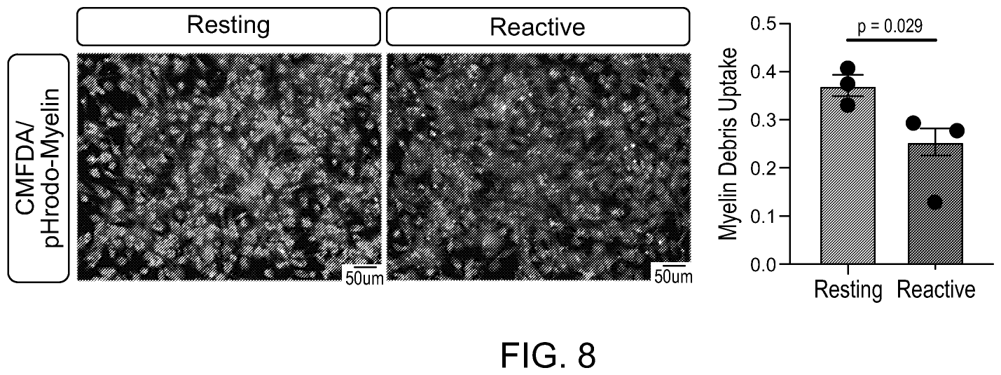

In particular, resting and reactive astrocytes were exposed to fluorescently labelled myelin debris. After 24 hrs, myelin debris phagocytosed by astrocytes was measured. The data shows that the resting astrocytes phagocytosed about 1.5-fold more labelled myelin debris when compared to that by reactive astrocytes (FIG. 8).

Resting astrocytes generated with the methods as described herein and also became reactive after infection with the Thieler's murine encephalomyelitis virus (TMEV). This shows that the methods described herein generate astrocytes that respond to viral infection.

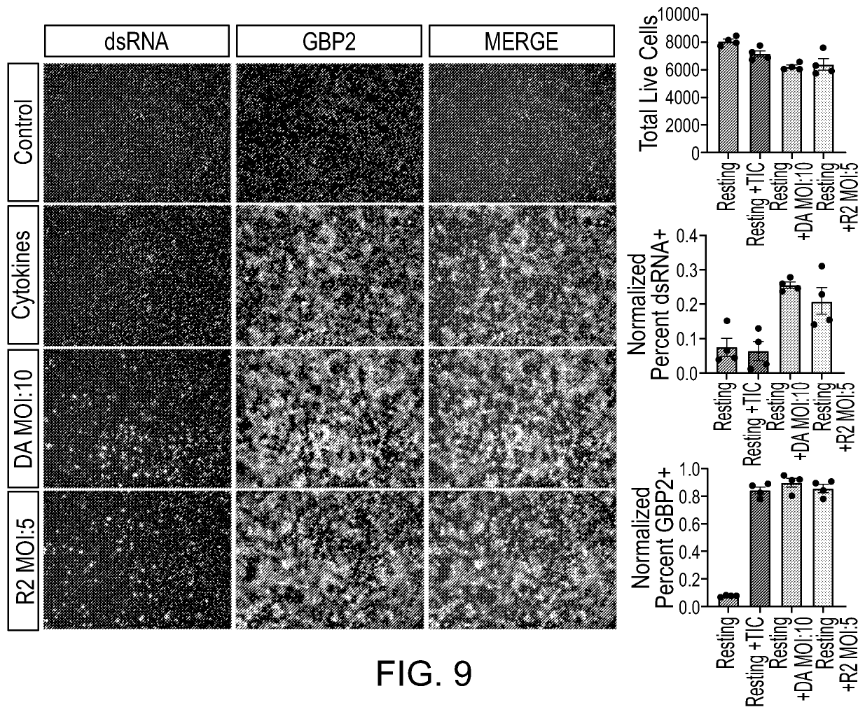

Specifically, resting astrocytes were exposed to TMEV at two different multiplicity of infection (MOI) for 1 hr. After 1 hr, virus was washed out before 24 hrs of incubation and staining for double-stranded RNA (dsRNA) to measure viral infection and the reactive astrocyte marker GBP2. The data showed that TMEV MOI of 5 and 10 increased dsRNA in resting astrocytes by about 1.5 to about 2 fold compared to resting astrocytes not infected with TMEV, indicating that the resting astrocytes have been infected (FIG. 9). Resting astrocytes infected with TMEV (DA or R2) expressed GBP2 at similar levels compared to that in resting astrocytes incubated with TNFα, IL1a, and/or C1q as reactive astrocyte drivers (TIC), but about 8 fold to 9 fold higher when compared to the negative control, uninfected resting astrocytes.

Reactive astrocytes further decreased the formation of mature oligodendrocytes in co-culture. This shows that reactive astrocytes generated with the methods described herein acquired a pathological function.

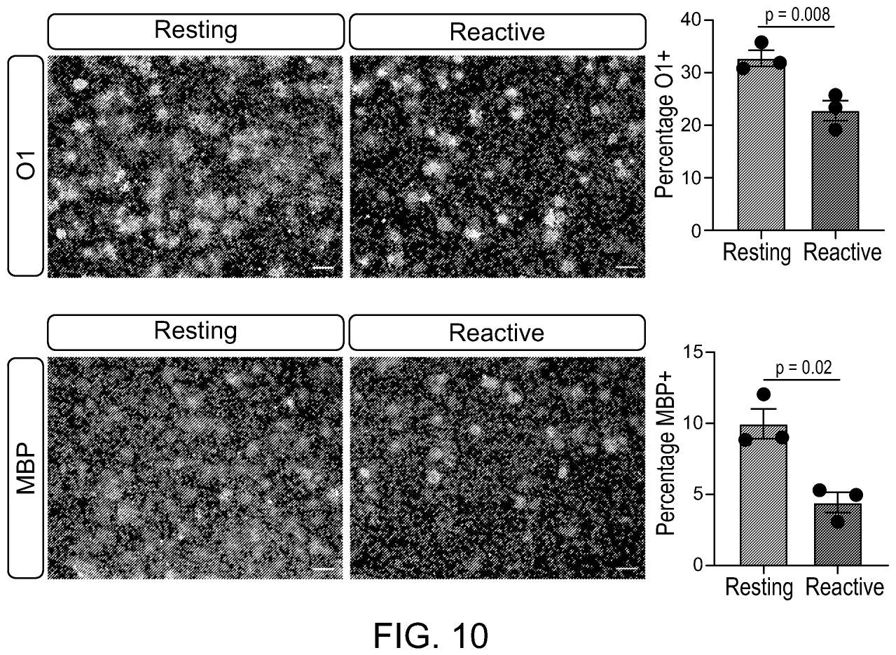

Resting or reactive astrocytes were co-cultured with differentiating oligodendrocytes for 48 to 72 hrs and then stained for the mature oligodendrocyte markers O1 or MBP (myelin basic protein) (FIG. 10). O1 and MBP expression, indicating maturation, by oligodendrocytes was about 1.5 fold and 2 fold less when co-cultured with reactive astrocytes compared to co-culturing with resting astrocyte.

### REFERENCES

Liddelow, S., Guttenplan, K., Clarke, L. et al. Neurotoxic reactive astrocytes are induced by activated microglia. Nature 541, 481-487 (2017).

All sited references and publications are incorporated herein by reference in their entirety, preferably at the instance where they are cited.

1. A chemically-defined, serum-free, resting astrocyte culture medium, for culturing resting astrocytes, the medium comprising:
i) a serum-free basal medium (such as DMEM/Neurobasal medium), wherein said basal medium (1) is devoid of significant source of proteins, lipids, or growth factors, and/or (2) comprises sufficient energy source, nitrogen source, carbon source, amino acids, vitamins, and inorganic salts to support growth of mammalian neuronal cells in the absence of feeder cells,
optionally, the serum-free basal medium further comprises (3) an amino acid supplement as a source of L-glutamine (such as GLUTAMAX™ brand of L-alanyl-L-glutamine dipeptide), such as about 1-3 mM (e.g., about 2 mM) L-alanyl-L-glutamine dipeptide; and/or a source of sodium pyruvate, such as about 0.5-1.5 mM (e.g., about 1 mM) sodium pyruvate,
ii) an antioxidant (e.g., including one that inhibits lipid peroxidation, such as sodium selenite);
iii) an iron carrier that regulates iron homeostasis (e.g., transferrin);
iv) a polyamine that promotes cell division (e.g., putrescine);
v) a hormone that activates the progesterone receptor (e.g., progesterone); and,
vi) a trophic factor that promotes astrocyte survival in culture;
wherein resting astrocytes cultured in said resting astrocyte culture medium exhibit a stellate morphology, express canonical mature astrocyte markers (e.g., AQP4, GLT-1, VIMENTIN, GLAST, and/or ALDH1L1), and/or do not express reactive astrocyte markers (e.g., Lcn2, Steap4, and Cxc110).

2. The resting astrocyte culture medium of claim 1, wherein the trophic factor that promotes astrocyte survival is a neurotrophic factor.

3. The resting astrocyte culture medium of claim 2, wherein the neurotrophic factor is selected from the group consisting of heparin binding EGF like growth factor (HBEGF), nerve growth factor (NGF), brain-derived neurotrophic factor (BDNF), neurotrophin-3 (NT-3), and ciliary neurotrophic factor (CNTF).

4. The resting astrocyte culture medium of claim 3, wherein the neurotrophic factor is HBEGF; optionally, about 2-10 ng/mL (e.g., about 5 ng/mL) HBEGF.

5. The resting astrocyte culture medium of any one of claims 1-4, wherein the antioxidant is selected from the group consisting of sodium selenite and N-acetylcysteine (NAC), catalase, reduced glutathione, alpha-tocopherol, and superoxide dismutase.

6. The resting astrocyte culture medium of claim 5, wherein the antioxidant comprises NAC and sodium selenite; optionally, about 20-40 μM (e.g., about 30 μM) NAC and/or about 5.2-40 ng/mL (e.g., about 10-20 ng/mL) sodium selenite.

7. The resting astrocyte culture medium of any one of claims 1-6, wherein the serum-free basal medium further comprises:
(3a) an amino acid supplement as a source of L-glutamine (such as GLUTAMAX™ brand of L-alanyl-L-glutamine dipeptide), such as about 1-3 mM (e.g., about 2 mM) L-alanyl-L-glutamine dipeptide; and/or,
(3b) a source of sodium pyruvate, such as about 0.5-1.5 mM (e.g., about 1 mM) sodium pyruvate.

8. The resting astrocyte culture medium of any one of claims 1-7, the serum-free basal medium further comprising (4) a growth factor that facilitates utilization of glucose and amino acids (e.g., insulin or insulin like growth factor 1 (IGF-1)); optionally, about 0-25 μg/mL insulin.

9. The resting astrocyte culture medium of any one of claims 1-8, comprising:
i) 1:1 mixture of DMEM/Neurobasal media mixture comprising sodium pyruvate and L-alanyl-L-glutamine dipeptide in the serum-free basal medium;
ii) sodium selenite and NAC as said antioxidant;
iii) transferrin as said iron carrier;
iv) putrescine as said polyamine;
v) progesterone as said hormone that activates the progesterone receptor; and,
vi) HBEGF as said trophic factor that promotes astrocyte survival in culture.

10. The resting astrocyte culture medium of any one of claims 1-9, comprising:
i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture;
ii) about 5.2-40 ng/mL (e.g., about 10-20 ng/mL) sodium selenite and about 20-40 μM (e.g., about 30 μM) NAC;
iii) about 50-200 μg/mL (e.g., about 100 μg/mL) transferrin;
iv) about 8-32 μg/mL (e.g., about 16 μg/mL) putrescine;
v) about 6-60 ng/mL (e.g., about 30 ng/mL) progesterone;
vi) about 0-25 μg/mL insulin; and,
vii) about 5-10 ng/mL (e.g., about 5 ng/mL) HBEGF.

11. A chemically-defined, serum-free, astrocyte enrichment and/or maturation culture medium, for astrocyte enrichment and/or maturation, the medium comprising the resting astrocyte culture medium of any one of claims 1-10, and further comprising:
viii) a TGF-beta superfamily cytokine that promotes astrogenesis and astrocyte maturation through SMAD dependent-signaling;
ix) an IL-6 superfamily cytokine that actives STAT signaling through leukemia inhibitory factor (LIF) receptor R (LIFRO) and/or glycoprotein 130 (gp130); and
x) a mitogen and trophic factor that promotes astrocyte survival and proliferation, as well as a resting/quiescent astrocyte state;
wherein said cell culture medium promotes enrichment and/or maturation for astrocytes in astrocyte-containing neuronal tissues.

12. The astrocyte enrichment and/or maturation culture medium of claim 11, wherein the factor to activate SMAD dependent-signaling is a transforming growth factor R (TGF-β) family member.

13. The astrocyte enrichment and/or maturation culture medium of claim 12, wherein the TGF-P family member is a bone morphogenetic protein (BMP).

14. The astrocyte enrichment and/or maturation culture medium of claim 13, wherein the BMP is selected from the group consisting of BMP2, BMP4, BMP5, BMP6, BMP7, BMP10 and BMP15.

15. The astrocyte enrichment and/or maturation culture medium of claim 14, wherein the BMP is BMP4; optionally, 5-100 ng/mL BMP4 (e.g., 5-20 ng/mL BMP4 for astrocyte enrichment in said astrocyte enrichment and/or maturation culture medium; and/or about 25-100 ng/mL BMP4 for astrocyte maturation in said astrocyte enrichment and/or maturation culture medium).

16. The astrocyte enrichment and/or maturation culture medium of any one of claims 11-15, wherein the IL-6 superfamily cytokine is IL-6, ciliary neurotrophic factor (CNTF), leukemia inhibitory factor (LIF), or oncostatin M (OSM).

17. The astrocyte enrichment and/or maturation culture medium of claim 16, wherein the IL-6 superfamily cytokine is CNTF; optionally, about 5-20 ng/mL (e.g., about 10 ng/mL) CNTF.

18. The astrocyte enrichment and/or maturation culture medium of any one of claims 11-17, wherein the mitogen and trophic factor is epidermal growth factor (EGF), basic fibroblast growth factor (bFGF), fibroblast growth factor 2 (FGF2), fibroblast growth factor 8 (FGF8), platelet derived growth factor (PDGF), and insulin like growth factor 1 (IGF-1).

19. The astrocyte enrichment and/or maturation culture medium of claim 18, wherein the mitogen and trophic factor is FGF2; optionally about 10-40 ng/mL (about 20 ng/mL) FGF2.

20. The astrocyte enrichment and/or maturation culture medium of any one of claims 11-19, comprising:
i) 1:1 mixture of DMEM/Neurobasal media mixture comprising sodium pyruvate and L-alanyl-L-glutamine dipeptide in the serum-free basal medium;
ii) sodium selenite and NAC as said antioxidant;
iii) transferrin as said iron carrier;
iv) putrescine as said polyamine;
v) progesterone as said hormone that activates the progesterone receptor;
vi) HBEGF as said trophic factor that promotes astrocyte survival in culture;
vii) BMP4 as the TGF-beta superfamily cytokine;
viii) CNTF as the IL-6 superfamily cytokine; and,
ix) FGF2 as the mitogen and trophic factor.

21. The astrocyte enrichment and/or maturation culture medium of any one of claims 11-20, which is astrocyte enrichment culture medium, comprising:
i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture;
ii) about 5.2-40 ng/mL (e.g., about 10-20 ng/mL) sodium selenite and about 20-40 μM (e.g., about 30 μM) NAC;
iii) about 50-200 μg/mL (e.g., about 100 μg/mL) transferrin;
iv) about 8-32 μg/mL (e.g., about 16 μg/mL) putrescine;
v) about 6-60 ng/mL (e.g., about 30 ng/mL) progesterone;
vi) about 0-25 μg/mL insulin;
vii) about 2-10 ng/mL (e.g., about 5 ng/mL) HBEGF;
viii) about 5-20 ng/mL (e.g., about 10 ng/mL) CNTF;
ix) about 10-40 ng/mL (about 20 ng/mL) FGF2; and,
x) about 5-20 ng/mL (e.g., about 10 ng/mL) BMP4.

22. The astrocyte enrichment and/or maturation culture medium of any one of claims 11-20, which is astrocyte maturation culture medium, comprising:
i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture;
ii) about 5.2-40 ng/mL (e.g., about 10-20 ng/mL) sodium selenite and about 100-300 μM (e.g., about 200 μM) NAC;
iii) about 50-200 μg/mL (e.g., about 100 μg/mL) transferrin;
iv) about 8-32 μg/mL (e.g., about 16 μg/mL) putrescine;
v) about 6-60 ng/mL (e.g., about 30 ng/mL) progesterone;
vi) about 0-25 μg/mL insulin;
vii) about 2-10 ng/mL (e.g., about 5 ng/mL) HBEGF;
viii) about 5-20 ng/mL (e.g., about 10 ng/mL) CNTF;
ix) about 10-40 ng/mL (about 20 ng/mL) FGF2; and,
x) about 25-100 ng/mL (e.g., about 50 ng/mL) BMP4.

23. A chemically-defined, serum-free, astrocyte activation culture medium, for transitioning astrocytes from resting state to activation state, the medium comprising the resting astrocyte culture medium of any one of claims 1-10, except for the trophic factor that promotes astrocyte survival, and further comprising one or more microglia-derived reactive astrocyte drivers (or proinflammatory cytokines) that promote the transition of astrocytes to a damaging reactive state.

24. The astrocyte activation culture medium of claim 23, wherein the reactive astrocyte drivers (or proinflammatory cytokines) comprise tumor necrosis factor alpha (TNFα), interleukin 1 alpha (IL1α), and/or complement component 1q (C1q).

25. The astrocyte activation culture medium of claim 23 or 24, comprising:
i) 1:1 mixture of DMEM/Neurobasal media mixture comprising sodium pyruvate and L-alanyl-L-glutamine dipeptide in the serum-free basal medium;
ii) sodium selenite and NAC as said antioxidant;
iii) transferrin as said iron carrier;
iv) putrescine as said mitogen;
v) progesterone as said hormone that activates the progesterone receptor; and,
vi) TNFα, IL1α, and/or C1q as said reactive astrocyte drivers.

26. The astrocyte activation culture medium of any one of claims 23-25, comprising:
i) about 0.5-1.5 mM (e.g., about 1 mM) sodium pyruvate and about 1-3 mM (e.g., about 2 mM) L-alanyl-L-glutamine dipeptide in 1:1 mixture of DMEM/Neurobasal media mixture;
ii) about 5.2-40 ng/mL (e.g., about 10-20 ng/mL) sodium selenite and about 20-40 μM (e.g., about 30 μM) NAC;
iii) about 50-200 μg/mL (e.g., about 100 μg/mL) transferrin;
iv) about 8-32 μg/mL (e.g., about 16 μg/mL) putrescine;
v) about 6-60 ng/mL (e.g., about 30 ng/mL) progesterone;
vi) about 0-25 μg/mL insulin; and,
vii) about 1-5 ng/mL (e.g., about 3 ng/mL) IL-1α, about 15-60 ng/mL (e.g., about 30 ng/mL) TNFα, and about 200-800 ng/mL (e.g., about 400 ng/mL) C1q.

27. A method of isolating a substantially pure culture of resting astrocytes from a population of astrocyte-containing cells, the method comprising:
(1) culturing single cells of the population of astrocyte-containing cells, in said astrocyte enrichment culture medium of any one of claims 11-21, with fresh medium change about every 2 days until a confluent culture is formed, in order to enrich for proliferating/proliferated astrocytes in the single cell culture;
(2) passaging the confluent culture once in said astrocyte maturation culture medium of any one of claims 11-20 and 22 for about 2 days to permit astrocyte maturation, or optionally cryopreserving the confluent culture before said passaging; and,
(3) replacing the astrocyte maturation culture medium after about 2 days, with said resting astrocyte culture medium of any one of claims 1-10 for about 3 more days,
thereby producing the substantially pure culture of resting astrocytes.

28. The method of claim 27, wherein the population of astrocyte-containing cells are derived from mammalian neuronal tissues, and differentiated or differentiating stem cells or progenitor cells.

29. The method of claim 28, wherein the mammalian neuronal tissues are isolated from a central nervous system (CNS) tissue, such as cortex, corpus callosum, hippocampus, midbrain, pons, medulla, brainstem, cerebellum, and/or spinal cord.

30. The method of claim 28, wherein said differentiated or differentiating stem cells or progenitor cells comprise oligodendrocyte progenitors (OPCs), glial progenitors (GPCs), glial restricted progenitors (GRPs), and/or neural progenitors (NPCs).

31. The method of claim 28 or 30, wherein the stem cells or progenitor cells comprise induced pluripotent stem cells (iPSCs), embryonic stem cells (ESCs), iPS cells, neural stem cells (NSCs), and/or epiblast stem cells (EpiSCs).

32. The method of any one of claims 27-29, wherein the astrocyte-containing cells are derived from mammalian neuronal tissues by chemical, enzymatic, and/or mechanical dissociation of the mammalian neuronal tissues.

33. The method of claim 32, wherein said dissociation comprises dissociating the mammalian neuronal tissues using enzymatic digestion with mechanical trituration.

34. The method of claim 33, wherein said enzymatic digestion is performed with papain, trypsin, dispase, and/or collagenase.

35. The method of any one of claims 27-34, wherein said resting astrocytes:
(1) are positive for any one or more of the astrocyte markers selected from GFAP, AQP4, GLT-1, VIMENTIN, GLAST, and ALDH1L1;
(2) do not express Lcn2, Steap4, and/or Cxc110;
(3) exhibit a stellate morphology; and/or
(4) only express mature astrocyte genes (such as Gja1 or Sox9), with no expression of oligodendrocyte genes (such as Sox10 and Mbp), microglia genes (such as Cd68 and Tmem119), and neuron markers (such as Nef1 and Snap25).

36. The method of any one of claims 27-35, wherein said resting astrocytes take up exogenous glutamate.

37. The method of claim 36, wherein said resting astrocyte take up at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of exogenous glutamate.

38. The method of any one of claims 27-37, wherein said resting astrocytes have increased phagocytic activity when compared to reactive astrocytes.

39. The method of claim 38, wherein said resting astrocytes have at least 1.2 fold, at least 1.3 fold, at least 1.4 fold, at least 1.5 fold, at least 1.6 fold, at least 1.7 fold, at least 1.8 fold, at least 1.9 fold, at least 2.0 fold increased phagocytic activity when compared to reactive astrocytes.

40. The method of any one of claims 27-39, wherein said resting astrocytes express one or more reactive astrocyte markers when infected with Thieler's murine encephalomyelitis virus (TMEV), thereby becoming reactive astrocytes.

41. The method of claim 40, wherein the reactive astrocyte marker is guanylate binding protein 2 (GBP2).

42. The method of claim 40 or claim 41, wherein said resting astrocytes infected with TMEV express at least 70%, at least 75%, at least 80%, at least 85%, at least 90%, at least 95%, or at least 98% more reactive astrocyte marker compared to uninfected resting astrocytes.

43. The method of any one of claims 27-42, wherein at least about 90%, 95%, 96%, 97%, 98%, 99% or more of the cells in the substantially pure culture are resting astrocytes.

44. The method of any one of claims 27-43, wherein the substantially pure culture of resting astrocytes comprises about 10⁶, 10⁷, 10¹, 10⁹, 10¹⁰, 10¹¹, 10¹² or 10¹³ resting astrocytes.

45. The method of any one of claims 27-44, further comprising, before culturing the single cells in said astrocyte enrichment culture medium (such as the astrocyte enrichment culture medium of claim 21), culturing the single cells for about 24 hours in a media consisting of Dulbecco's Modified Eagle Medium/Nutrient Mixture F-12 (DMEM/F-12), N-2 Supplement, B-27 Supplement, GLUTAMAX™ Supplement, Penicillin-Streptomycin, and FGF-2.

46. A cell culture comprising a population of astrocyte-containing cells cultured in the culture medium of any one of claims 1-26.

47. The cell culture of claim 46, wherein the culture medium is
(1) the chemically-defined, serum-free, astrocyte enrichment culture medium of any one of claims 11-21;
(2) the chemically-defined, serum-free, astrocyte maturation culture medium of any one of claims 11-20 and 22; or,
(3) the chemically-defined, serum-free, the resting astrocyte culture medium of any one of claims 1-10.

48. A cryopreserved culture of enriched astrocytes, obtained from a population of astrocyte-containing cells by:
(a) culturing single cells of the population of astrocyte-containing cells, in said astrocyte enrichment culture medium of any one of claims 11-21, with fresh medium change about every 2 days until a confluent culture is formed, in order to enrich for proliferating/proliferated astrocytes in the single cell culture; and,
(b) cryopreserving the confluent culture.

49. A cryopreserved substantially pure culture of resting astrocytes, wherein the substantially pure culture of resting astrocytes are obtained by the method of any one of claims 27-45.

50. The cryopreserved culture of claim 48 or 49, wherein the population of astrocyte-containing cells are derived from mammalian neuronal tissues, and differentiated or differentiating stem cells or progenitor cells.

51. The cryopreserved culture of claim 50, wherein the mammalian neuronal tissues are isolated from a central nervous system (CNS) tissue, such as cortex, corpus callosum, hippocampus, midbrain, pons, medulla, brainstem, cerebellum, and/or spinal cord.

52. The cryopreserved culture of claim 50, wherein said differentiated or differentiating stem cells or progenitor cells comprise oligodendrocyte progenitors (OPCs), glial progenitors (GPCs), glial restricted progenitors (GRPs), and/or neural progenitors (NPCs).

53. The cryopreserved culture of claim 50 or 51, wherein the stem cells or progenitor cells comprise induced pluripotent stem cells (iPSCs), embryonic stem cells (ESCs), neural stem cells (NSCs), iPS cells, and/or epiblast stem cells (EpiSCs).

54. A method of identifying a compound that inhibits reactive astrocyte formation from resting astrocytes, the method comprising: contacting a substantially pure culture of resting astrocytes obtained by the method of any one of claims 27-45, or a thawed culture of the cryopreserved substantially pure culture of resting astrocytes of any one of claims 49-53, with a candidate compound from a library of compounds, in the presence of the astrocyte activation culture medium of any one of claims 23-26, for a sufficient period of time (e.g., 24 hours), before determining the expression level of a marker gene for reactive astrocyte; wherein the candidate compound that statistically significantly decreased the number of marker gene-positive reactive astrocytes by greater than 50%, 60%, 70%, 80%, 90%, 95% or more compared to vehicle (e.g., a solvent for the candidate compound such as DMSO) control is identified as the compound that inhibits reactive astrocyte formation.

55. The method of claim 54, wherein the marker gene comprises one or more of GBP2 PSMB8, C3, H2-D1, H2-T23, SERPING1, and IIGP1 (e.g., GBP2).

56. The method of claim 53 or 54, further comprising determining the expression level of the marker gene.

57. The method of any one of claims 54-56, wherein the candidate compound is not significantly toxic to astrocytes (e.g., the candidate compound does not decrease the counted number of live cells by greater than 30% compared to the vehicle control).

58. The method of any one of claims 54-57, wherein the method has a z-prime score of 0.6, 0.7 or higher.

59. The method of any one of claims 54-58, wherein the resting astrocytes are contacted in 384-well tissue culture plates in a high throughput platform suitable for multiplex screening.

60. The method of any one of claims 54-59, wherein the resting astrocytes are contacted by the candidate compound prior to (e.g., 1 hour prior to), simultaneously with, or subsequent to (e.g., within 30 min of) contacting with the astrocyte activation culture medium.

61. The method of any one of claims 54-60, wherein the astrocyte activation culture medium comprises about 1-5 ng/mL (e.g., about 3 ng/mL) IL-1α, about 15-60 ng/mL (e.g., about 30 ng/mL) TNFα, and about 200-800 ng/mL (e.g., about 400 ng/mL) C1q.

62. The method of any one of claims 54-61, further comprising confirming that the candidate compound does not substantially affect (e.g., decrease) the expression level of a pan-astrocyte marker (such as vimentin).

63. The method of any one of claims 54-61, wherein the library of compounds comprise histone deacetylase (HDAC) inhibitors, proteasome inhibitors, and inhibitors of NFκB signaling.
